(19) United States
(12) Patent Application Publication
(10) Pub. No.: US 2009/0215080 A1
(43) Pub. Date: Aug. 27, 2009

(54) METHODS FOR IDENTIFICATION OF INHIBITORS OF ENZYME ACTIVITY

(75) Inventors: Sukanto Sinha, San Francisco, CA (US); Tamie Jo Chilcote, San Francisco, CA (US)

Correspondence Address: BELL & ASSOCIATES, 58 West Portal Avenue No. 121, SAN FRANCISCO, CA, 94127, US

(73) Assignee: ActiveSite Pharmaceuticals, South San Francisco, CA (US)

(21) Appl. No.: 12/083,428

(22) PCT Filed: Oct. 12, 2006
(86) PCT No.: PCT/US2006/040256

§ 371 Date: Feb. 2, 2009

Related U.S. Application Data
(60) Provisional application No. 60/726,692, filed on Oct. 13, 2005.
(60) Provisional application No. 60/738,871, filed on Nov. 21, 2005.

(51) Int. Cl. G01N 33/573

(52) U.S. Cl. 435/7.4

(57) ABSTRACT

The invention discloses compositions and methods of synthesis to create novel ligands and drugs and identifying such compounds as inhibitors of enzyme targets for use in the treatment of clinical disorders, including cancer, infectious diseases, parasitic infestations, neurological disorders, reproductive disorders, inflammatory disorders, circulatory disorders, and metabolic disorders.

### TECHNICAL FIELD

This invention encompasses a method for identifying a compound having binding activity for an enzyme, compositions, methods of synthesizing said compositions and methods of using said compositions to inhibit enzymes.

### BACKGROUND ART

Small molecule inhibitors of enzyme activity have been used in science and medicine for several centuries. Aspirin (acetylsalicylate), derived as an extract from willow bark, has been used in Europe reputedly since the time of Hippocrates for a multiplicity of ailments, most notably as an antipyretic (Stone E. (1763) Philos. Trans. R. Soc. Lond. 53: 195-200; Dreser H. (1899) Pfluger's Arch. 76: 306-318). In general, small molecule inhibitors are analogues of a substrate or co-factor that interacts with the enzyme. Examples of these are peptide fragments of the naturally occurring protein kinase A inhibitor (PKI) of cyclic-AMP-dependent protein kinase (PK-A) and H7 inhibitor (an ATP analogue), an inhibitor of protein kinase C(PK-C) activity.

Many receptor- or enzyme-interacting drugs have been developed that are (i) a derivative of a naturally-occurring compound, (ii) that are analogues of such naturally-occurring compounds, or (iii) that apparently mimic the naturally-occurring compound, either by having a similar chemistry to enable them to interact within the active site of the receptor or enzyme or by occupying the same electrostatic space as the naturally-occurring compound. Some examples of these are synthetic steroid hormones, analogues of nucleosides such as azidothymidine, and the endorphin mimics such as opiates. Other drugs can inhibit or activate an enzyme in a non-competitive manner, such as by binding to an activation domain that itself regulates the enzyme activity, such as the well-understood interactions between phorbol ester and PK-C.

In the past, such drug molecules have been selected upon their propensity for binding to the target of interest, be it a receptor or an enzyme. In the main, these drugs were originally identified by serendipity, often having been used as part of a crude extract in traditional medicine. Only later was the active ingredient identified and either used in pure form or synthesized from chemical precursors. Due to their natural origins, they were pre-selected for stability in an inert carrier, were resistant to catalysis in the gut, and were more likely to be metabolized slowly by the liver.

The approach in the past, as well as in current times, to select and create new compounds for use as drugs, tends to follow the following steps. (1) Tens of thousands of randomly synthesized organic compounds are screened against an array of different drug targets. (2) Those that bind (competitively or non-competitively) are then selected for further analysis and modification. (3) The process of modification generally results in large molecules. Frequently these molecules do not conform to “Lipinski's rule of five” which was developed in the 1990s to select for molecules that would most efficiently transport across the gut epithelium. The rule comprises the following parameter limits: mass <500 daltons (Da), logP (calculated octanol/water partition coefficient: lipophilicity) <5.0, hydrogen bond donors <5, hydrogen bond acceptors <10. (See Lipinski, C. A., Lombardo, F., Dominy, B. W., Feeney, P. J.; (1997) Adv. Drug Deliv. Rev. 23: 3-25.) However, optimal drug discoveiy has been difficult where the compounds to be screened are in the size range of about 200 Da.

Traditional high throughput screening results in percentage inhibition of enzyme activity at maximum concentrations of between 10 and 30 μM. However, such screening strategies have a low “hit rate”, that is, 100,000 random compounds need to be screened in order to obtain at least one or two useful leads. In addition, these methods do not appear to work well for many important targets (for example, β-secretase/β-site amyloid precursor protein cleaving enzyme; BACE) and cannot detect low affinity ligands due to solubility limitation of the potential ligand above 30 μM. Analysis of “false hits” (ligands that appear to bind but are artifacts of the method) contributes to major costs and time delays in lead discovery and optimization procedures.

New parameters for identifying ligands have recently been suggested by Hopkins et al. (Hopkins et al. (2004) Drug Discovery Today 9: 430-431). These parameters define the “ligand efficiency” and have the following properties:

ΔG=−RT ln K_{d}

Maximum affinity per non-H atom=−1.5 kcal

Ligand efficiency=(ΔG/n·non-H atoms)÷(−1.5 kcal)
Thus, a “Lipinski” drug (500 Da, Kᵢ=10 nM) comprising 38 non-H atoms will have a “ligand efficiency” of −0.29 kcal·atom⁻¹=19% of the maximum affinity.

Compounds of about 200 Da are usually weak binders and hence are weak inhibitors. Such small compounds are usually analysed using X-ray diffraction of crystallized inhibitors with the target enzyme, but this too is limited by solubility and is expensive. There is a need in the art for a method to rapidly measure binding activity and to obtain structure activity relationship (SAR). More particularly there is a need for a catalysis-based approach that is between one thousand to ten thousand times more sensitive that competitive inhibition.

Enzymes such as proteases and kinases recognize their protein substrates using a combination of multiple pieces of information derived from the substrate—which will be referred to as Specificity Determinants (SD). Generally, one or more Primary SD (denoted by P_{SD}), when found in combination with one or more Secondary SD (S_{SD}), leads to the recognition of a given peptide or protein as a substrate. An example of the concept of P_{SD} and S_{SD} can be made by considering the serine protease coagulation Factor Xa (activated Factor X). Factor Xa specifically recognizes and cleaves an amide bond carboxy-terminal to the amino acid arginine (Arg) in a protein or peptide substrate, while it will not do so if other amino acid residues are substituted in place of Arg. Thus Arg can be considered to be an essential P_{SD} for Factor Xa. However, this cleavage takes place efficiently when the three amino acids amino-terminal to the Arg have the linear sequence Ile-Glu-Gly, such as in the chromogenic peptide substrate Bz-Ile-Glu-Gly-Arg-para-nitro-anilide (Bz-Ile-Glu-Gly-Arg-pNA) (Brown et al, (2004) Handbook of Proteolytic Enzymes, 2^{nd} Ed., pp 1662-1666, Elsevier). If this tripeptide sequence is deleted from the synthetic substrate, as in Bz-Arg-pNA, the truncated peptide will be only poorly cleaved by Factor Xa in spite of the constant presence of the P_{SD} Arg. Thus, the tripeptide sequence Ile-Glu-Gly could be considered a S_{SD} for Factor Xa. The closely related serine protease thrombin, while sharing the exact P_{SD} as factor Xa, has different S_{SD} requirements, and thus will catalyze the cleavage of different substrates than that for Factor Xa, especially those having a proline (Pro) residue immediately preceding the Arg residue.

Enzymes such as protein kinases also have P_{SD} and S_{SD} requirements. The P_{SD} for many serine/threonine (Ser/Thr) kinases is determined by the amino acid residue immediately C-terminal to the phosphorylatable Ser/Thr residue (Songyang et al, (1996) Mol. Cell. Biol., 16: 6486). For an important subfamily of Ser/Thr kinases—referred to as proline-directed kinases—a proline (Pro) residue at this P₊₁ position (the amino acid immediately C-terminal to the P₀ Ser/Thr) is either absolutely required, or highly preferred by such kinases. So, the P_{SD} for the proline-directed cyclin-dependent kinase cdk2 can be represented by Ser/Thr-Pro. In contrast, the extensively-studied cAMP-dependent protein kinase A (PKA) will not phosphorylate Ser/Thr-Pro motifs, instead requiring a Leu/Ile at the P₊₁ position. Therefore, its P_{SD} can be represented by Ser/Thr-Leu/Ile.

Neither of these kinases, though, will efficiently phosphorylate a Ser-Pro or a Ser-Ile dipeptide—additional S_{SD}, usually provided by the amino acid sequences flanking the P₀-P₊₁ residues (Songyang et al, supra), is required to have an efficiently phosphorylatable substrate.

Of note a recent paper by Wood et al. discloses methods for identification of nonpeptidic protease inhibitors. The method relies on hydrolysis of a peptide (amide) bond in a chimeric substrate comprising organic moieties and N-acyl aminocoumarins (fluorogenic) (Wood et al. (2005) J. Am. Chem. Soc. 127: 15521-15527).

Dipeptidyl peptidase-IV (DPP-IV) is a serine protease that cleaves N-terminal dipeptides from a peptide chain containing, preferably, a proline residue in the penultimate position. Although the role of DPP-IV in mammalian systems has not been completely established, it is believed to play an important role in neuropeptide metabolism, T-cell activation, attachment of cancer cells to the endothelium, and the entry of HIV into lymphoid cells.

Several previous patents disclose methods of solid surface chimeric peptide synthesis; see for example, U.S. Pat. Nos. 6,699,871, 6,395,767, 6,617,340, 6,110,949, 6,011,155, WO2005044195, WO2004112701, WO2004069162, WO2004050022, WO2004043940, WO2005012249, all of which disclose inhibitors of dipeptidyl peptidase-IV enzymes. International Patent Application No. WO2004103272 discloses thiol and disulfide-containing maytansinoids used to conjugate with and to enhance cytotoxicity of cell-binding compounds. International Patent Application No. WO2004044195 discloses agmatine coumaryl transferase that catalyzes the first step in the biosynthesis of antimicrobial hydroxycinnamoylagmatine derivatives.

In spite of the foregoing known and disclosed systems, none of the above prior art disclose the methods of the instant invention.

Target-based drug discovery is dependent on the availability of two essential components—one or more “druggable” targets, and one or more lead molecules that can be the starting point for a lead optimization and drug-discovery campaign.

As a consequence of the enormous progress in determining the human genome, the number of potentially druggable cellular targets has been estimated to be between 600-1500, a very large number even if it turns out to be on the lower end of the estimate (Hopkins and Groom (2002) Nat. Rev. Drug Disc., 1: 727). Target availability is therefore not a relevant issue, even if the biology of many of these potential targets remains to be fully understood.

However, in spite of tremendous progress in automated screening technologies, for example, high-throughput screening (HTS) over the last 15 years, and the application of large, multi-million compound screening campaigns against targets of potentially high therapeutic value, decreasing number of new small molecule drugs are being approved by the FDA every year. The reasons for this are many, but at least one of the factors has to do with the essential nature of HTS: compound collections are screened at an arbitrary pre-set concentration usually not exceeding 10 μM (so as not to exceed the limits of compound solubility, and minimize non-specific effects that often predominate at higher compound concentrations) and “hits” are determined by “% inhibition”. Compounds that may interact with the target with a dissociation constant (K_{d})>30 μM, even if present in the collection, would thus be difficult to detect. In addition, the average MW of compounds present in random chemical collections tend to be in the 350-450 range, so even if a validated “hit” were to be obtained, say with a K_{d}˜10 μM and a MW of 400 Da, a lead-optimization effort focused on increasing potency towards the target often ends up with potent (<100 nM) but large (>500 Da) inhibitors that break the empirically derived Lipinski Rules of Five as described above and thus have the high lipophilicity, poor permeability and/or solubility that usually characterize non-drug-like molecules (Lipinski et al. (1997) Advanced Drug Delivery Reviews, 23: 3).

It has been suggested, and increasingly accepted, that one way out of this predicament would be to have methods to rapidly identify small (MW 200-300 Da) lead-like molecules that bind productively to the target of interest (Teague et al. (1999) Angew. Chem. Int. Ed., 38: 3743). Thus, if one is starting from a smaller lead molecule, it is less likely that the optimization process will generate a non-drug-like molecule, since it should be easier to keep within the Lipinski limits and generate a potent inhibitor with suitable pharmacokinetic-pharmacodynamic (PK-PD) properties. The difficulty is that molecules this small often have low affinity for the target, Ki>100 μM, which is difficult to measure by competitive inhibition methods for a variety of practical reasons (poor solubility and non-specific effects that dominate at high compound concentrations).

One approach that has been used to circumvent these intrinsic limitations has been to utilize a “library” of small (<250 Da) drug-like “fragments”, which are then soaked at high concentrations (>1 mM) with crystals of a target protein. Those molecules that bind, even weakly, are then identified by solving the crystal structure, which also allows determination of the mode of binding (Hartshorn et al. (2005) J. Med. Chem., 48: 403). Such small fragments are then chemically elaborated to provide binders with sufficient affinity to measure by traditional screening methods (for example, measuring inhibition of the enzyme target's activity), and thus may serve as the foundation for a hit-to-lead optimization campaign for a given target. However, this approach is technically complex, is limited to targets that crystallize relatively easily, has the potential of a high false positive rate, and is not applicable to compounds that are insoluble at high concentrations.

A generally applicable method that can be used to rapidly and efficiently screen for weakly interacting small molecules for many targets would therefore be valuable in obtaining potential starting points for lead identification and optimization. The invention described herein offers such a method, adaptable to enzyme targets from multiple therapeutically important classes.

### DISCLOSURE OF INVENTION

The invention is drawn to a method of identifying and synthesizing compositions used to inhibit the activity of an enzyme, in particular compositions having inhibitory activity against proteases, kinases, phosphatases, transferases, oxidoreductases, nucleotidases, hydrolases, lyases, and isomerases, for treatment of cancer, reproductive disorders, neurological disorders, and infectious disease.

In one embodiment the invention provides a method for identifying a compound having binding activity for an enzyme, the method comprising the steps of: (i) providing an enzyme; (ii) incubating a first sample of the enzyme with a first substrate thereby creating a first incubate, the first substrate comprising at least one R-group and wherein the enzyme catalyses conversion of the first substrate into a first product; (iii) measuring the increase in product formation, (iv) converting the rate of increase of product formation into a rate of first substrate catalysis by the enzyme (Rate_{first}); (v) incubating a second sample of the enzyme with a second substrate thereby creating a second incubate, the second substrate comprising at least one P_{SD} but not an R-group, and wherein the enzyme catalyses conversion of the second substrate into a second product; (vi) measuring the change in second product formation, (vii) converting the change of product formation into a rate of second substrate catalysis by the enzyme (Rate_{second}); (viii) determining the k_{cat}/Kₘ ratio of the first substrate (k_{cat}/Kₘ first); (viii) determining the k_{cat}/Kₘ ratio of the second substrate (k_{cat}/Kₘ second); (ix) determining the relative catalytic efficiency (RCE) of the R group; wherein a first substrate with a RCE>2 is identified as a compound having binding activity for the enzyme. In a preferred embodiment, the enzyme is selected from the group consisting of proteases, kinases, phosphatases, hydrolases, oxidoreductases, isomerases, transferases, methylases, acetylases, ligases, lyases, and the like. In another preferred embodiment, the P_{SD} comprises a moiety selected from the group consisting of an amino acid residue, an oligopeptide, a saccharide, a polysaccharide, a lipid, a phospholipid, a fatty acid, a glycoproterin, a proteoglycan, an aminoglycan, an alcohol amine, a nucleoside, a nucleotide, an oligonucleotide, a glycosyl phosphatidyl inositol, a steroid, and the like. In another preferred embodiment the second substrate further comprises a moiety selected from the group consisting of an amino acid residue, an oligopeptide, a saccharide, a polysaccharide, a lipid, a phospholipid, a fatty acid, a glycoproterin, a proteoglycan, an aminoglycan, an alcohol amine, a nucleoside, a nucleotide, an oligonucleotide, a glycosyl phosphatidyl inositol, a steroid, and the like. In a yet further preferred embodiment, measuring the rate of increase of product formation is selected from the group consisting of using analytical means to measure change in optical density of the incubate, colorimetry, fluorimetry, mass-spectroscopy, radioisotope analysis, pH analysis, phase partition of product, and electrochemical analysis of product. In a still further preferred embodiment, the first substrate further comprises a bond selected from the group consisting of an amide bond, a peptide bond, a covalent bond, a double bond, a triple bond, a keto bond, an oxo bond, a disulfide bond, and a phosphate bond. In one embodiment the incubate is incubated at a temperature from about 4° C. to about 75° C., preferably from about 10° C. to about 60° C., more preferably from about 15° C. to about 45° C., and most preferably the temperature is from about 16° C. to about 25° C. In one preferred embodiment the RCE is determined using the formula RCE=(k_{cat}/Kₘ first)/(k_{cat}/Kₘ second).

The invention also provides a method for identifying a compound having inhibitory activity for an enzyme, the method comprising the steps of: (i) providing an enzyme; (ii) incubating a first sample of the enzyme with a first substrate thereby creating a first incubate, the first substrate comprising at least one R-group and wherein the enzyme catalyses conversion of the first substrate into a first product; (iii) measuring the increase in first product formation, (iv) converting the rate of increase of product formation into a rate of first substrate catalysis by the enzyme (Rate_{first}); (v) providing a second substrate, the second substrate comprising at least one P_{SD} but not an R-group; (vi) incubating a second sample of the enzyme with the second substrate thereby creating a second incubate, wherein the enzyme catalyses conversion of the second substrate into a second product; (vii) measuring the increase in second product formation in the second incubate; (viii) converting the rate of first product formation into a rate of first substrate catalysis by the enzyme in the presence of second substrate (Rate_{second}); (ix) incubating a compound and another sample of the second substrate thereby creating a third incubate, the compound comprising at least one R-group; (x) adding a third sample of the enzyme to the third incubate; (xi) incubating the third incubate; (xii) measuring the increase in second product formation in the third incubate; (xiii) converting the rate of second product formation into a rate of second substrate catalysis by the enzyme in the presence of the compound (Rate_{compound}); (xiv) determining the k_{cat}/Kₘ ratio of the first substrate (k_{cat}/Kₘ first); (xv) determining the k_{cat}/Kₘ ratio of the second substrate (k_{cat}/Kₘ second); and (xvi) determining the relative catalytic efficiency (RCE) of the R-group; wherein the first substrate and the compound have at least one R-group in common, and wherein an R-group having a RCE>2 is identified as a compound having inhibitory activity for the enzyme.

In one alternative embodiment the invention provides a method for identifying a compound having inhibitory activity for an enzyme, the method comprising the steps of (i) providing an enzyme; (ii) incubating a first sample of the enzyme with a first substrate thereby creating a first incubate, the first substrate comprising at least one R-group and wherein the enzyme catalyses conversion of the first substrate into a first product; (iii) measuring the increase in product formation, (iv) converting the rate of increase in product formation into a rate of first substrate catalysis by the enzyme (Rate_{first}); (v) incubating a second substrate thereby creating a second incubate; (vi) adding a second sample of the enzyme to the second incubate; (vii) incubating the second incubate and wherein the enzyme catalyses conversion of the second substrate into a second product; (viii) measuring the increase in product formation; (ix) converting the rate of increase in product formation into a rate of second substrate catalysis by the enzyme (Rate_{second}); (x) incubating a compound and the second substrate thereby creating a third incubate, the compound comprising at least one R-group; (xi) adding a third sample of the enzyme to the third incubate; (xii) incubating the third incubate; (xiii) measuring the increase in product formation in the third incubate; (xiv) converting the rate of product formation into a rate of second substrate catalysis by the enzyme in the presence of compound (Rate_{compound}); (xv) determining the inhibition constant (Kᵢ) of the compound using the formula: K; =[compound]/((Rate_{second}/Rate_{compound})−1); (xvi) determining the k_{cat}/Kₘ ratio of the first substrate (k_{cat}/Kₘ first); (xvii) determining the relative k_{cat}/Kₘ ratio of the first substrate with reference to a substrate that does not contain an R-group (k_{cat}/Kₘ not R); (xviii) determining the relative catalytic efficiency (RCE) of the R group using the formula: RCE=(k_{cat}/Kₘ first)/(k_{cat}/Kₘ not R); wherein the first substrate and the second substrate have at least one amino acid residue in common, wherein the first substrate and the compound have at least one R-group in common, wherein the R group of the compound is less than 500 daltons (Da) in size, has logP<5.0, has hydrogen bond donors <5, has hydrogen bond acceptors <10, and wherein a compound having a Kᵢ<85 μM with an R-group found in a first substrate with a relative catalytic efficiency of at least 2 compared with the first substrate is identified as a compound having inhibitory activity for the enzyme. In one preferred embodiment the compound comprises an R group having a size less than 300 Da, has hydrogen bond donors <2, and has hydrogen bond acceptors <5. In another preferred embodiment, the enzyme is selected from the group consisting of proteases, kinases, phosphatases, hydrolases, oxidoreductases, isomerases, transferases, methylases, acetylases, ligases, and lyases. In a more preferred embodiment, the enzyme is a protease or a kinase. In a most preferred embodiment, the enzyme is a protease.

The invention also provides a method for identifying a small organic molecule (R group) having binding activity to a target enzyme, the method comprising the steps of: (a) synthesizing a compound comprising an R group and a synthetic peptide (SP) having the general formula R-SP or SP—R, the R group and the synthetic peptide linked using a covalent bond, wherein the synthetic peptide portion of the resulting synthetic compound molecule comprises a P_{SD} for the target enzyme, the synthetic peptide comprising at least one amino acid residue (Aaa) and one amide bond; (b) mixing the synthetic compound with the target enzyme under conditions that allow the target enzyme to have sufficient catalytic activity upon the synthetic compound; (c) measuring the amount of product generated; (d) determining the rate of product formation (Rate_{synthetic compound}); (e) comparing the rate of product formation with a rate of product formation generated from a reaction comprising the target enzyme and another substrate (Rate_{other substrate}), the other substrate selected from the group consisting of a natural substrate, a chromogenic substrate, a fluorogenic substrate, and a modified substrate; and (f) determining the RCE of the synthetic compound by RCE=(Rate_{synthetic compound}/Rate_{other substrate}), wherein if the RCE is >2, the small organic molecule R group is identified as an active-site binder of the target enzyme. In a preferred embodiment, the R group is selected from an organic molecule having at least one carboxylic acid group. In another preferred embodiment, the R group is selected from an organic molecule having at least one primary or secondary amino group. In another preferred embodiment, the SP is of the form H-Aaa-para-nitroanilide (H-Aaa-pNA), where Aaa is selected from the group consisting of any one of the twenty naturally occurring L-amino acids, a synthetic amino acid, an unnatural amino acid, and a chemically modified L-amino acid. In another preferred embodiment, the SP is of the form H-Aaa-7-amido-4-methylcoumarin (H-Aaa-AMC), where Aaa is selected from the group consisting of any one of the twenty naturally occurring L-amino acids, a synthetic amino acid, an unnatural amino acid, and a chemically modified L-amino acid. In a still further preferred embodiment, the SP is of the form H-Aaa-7-amido-4-trifluoromethylcoumarin (H-Aaa-AFC), where Aaa is selected from the group consisting of any one of the twenty naturally occurring L-amino acids, a synthetic amino acid, an unnatural amino acid, and a chemically modified L-amino acid. In an alternative embodiment, the SP is of the form H-Aaa₁-Aaa₂-X, where Aaa₁ and Aaa₂ are selected from the group consisting of any one of the twenty naturally occurring L-amino acids, a synthetic amino acid, an unnatural amino acid, and a chemically modified L-amino acid, and X is selected from the group consisting of OH, NH₂, pNA, AMC, and AFC. In another alternative embodiment, the SP is of the form H-Aaa₁-Aaa₂-Aaa₃-X, where Aaa₁, Aaa₂, and Aaa₃ are selected from the group consisting of any one of the twenty naturally occurring L-amino acids, a synthetic amino acid, an unnatural amino acid, a chemically modified L-amino acid, and a D-amino acid, and X is selected from the group consisting of OH, NH₂, pNA, AMC, and AFC. In a further alternative embodiment, the SP is of the form H-Aaa₁-Aaa₂-Aaa₃-Aaa₄-X, where Aaa₁, Aaa₂, Aaa₃, and Aaa₄ are selected from the group consisting of any one of the twenty naturally occurring L-amino acids, a synthetic amino acid, an unnatural amino acid, a chemically modified L-amino acid, and a D-amino acid, and X is selected from the group consisting of OH, NH₂, pNA, AMC, and AFC. In a yet further alternative embodiment, the SP is of the form Aaa₁-Aaa₂-Aaa₃-Aaa₄-Aaa₅-X, where Aaa₁, Aaa₂, Aaa₃, Aaa₄, and Aaa₅ are selected from the group consisting of any one of the twenty naturally occurring L-amino acids, a synthetic amino acid, an unnatural amino acid, a chemically modified L-amino acid, and a D-amino acid, and X is selected from the group consisting of OH, NH₂, pNA, AMC, and AFC. In one more preferred embodiment, the SP is of the form Y-Aaa₁-Aaa₂-OH, where Aaa₁ and Aaa₂ are selected from the group consisting of any one of the twenty naturally occurring L-amino acids, a synthetic amino acid, an unnatural amino acid, and a chemically modified L-amino acid, and Y is selected from the group consisting of H and CH₃C(═O). In a still further more preferred embodiment, the SP is of the form Y-Aaa₁-Aaa₂-Aaa₃-OH, where Aaa₁, Aaa₂, and Aaa₃ are selected from the group consisting of any one of the twenty naturally occurring L-amino acids, a synthetic amino acid, an unnatural amino acid, and a chemically modified L-amino acid, and Y is selected from the group consisting of H and CH₃C(═O). In another more preferred embodiment, the SP is of the form Y-Aaa₁-Aaa₂-Aaa₃-Aaa₄-OH, where Aaa₁, Aaa₂, Aaa₃, and Aaa₄ are selected from the group consisting of any one of the twenty naturally occurring L-amino acids, a synthetic amino acid, an unnatural amino acid, and a chemically modified L-amino acid, and Y is selected from the group consisting of H and CH₃C(═O). In a still further more preferred embodiment, the SP is of the form Y-Aaa₁-Aaa₂-Aaa₃-Aaa₄-Aaa₅-OH, where Aaa₁, Aaa₂, Aaa₃, Aaa₄, and Aaa₅, are selected from the group consisting of any one of the twenty naturally occurring L-amino acids, a synthetic amino acid, an unnatural amino acid, and a chemically modified L-amino acid, and Y is selected from the group consisting of H and CH₃C(═O). In a more preferred embodiment the target enzyme is a protease. In an alternative more preferred embodiment, the target enzyme is a protein kinase. In a still further alternative more preferred embodiment, the target enzyme is a protein tyrosine phosphatase. In another alternative preferred embodiment, the target enzyme is a proline hydroxylase. In a yet other alternative embodiment, the target enzyme is a histone deacetylase. In one preferred embodiment the Aaa is selected from the group consisting of: Arg, Lys, Pro, Asp, Val, Cys, and Tyr. In another more preferred embodiment, the synthetic peptide is of the form H-Ser-Pro-Lys-X and where X is OH or NH₂. In another more preferred embodiment the synthetic peptide is of the form Y-Lys-Ser-Pro-OH and where Y is H or CH₃C(═O).

The invention also provides a method for synthesizing a compound having at least one primary specific determinant (P_{SD}) and at least one secondary specific determinant (S_{SD}) and having binding activity to the active site of an enzyme the method comprising the steps of (i) providing a enzyme; (ii) incubating the enzyme with a substrate of the enzyme under appropriate reaction conditions, the substrate having at least one P_{SD} and at least one S_{SD}; (iii) measuring the amount of product formed; (iv) identifying said substrate having a relative k_{cat}/Kₘ ratio of at least 2 (compared to a second generation substrate); (v) removing the S_{SD} from the substrate thereby creating a second generation substrate with at least one amine group; (vii) reacting the amine group of the second generation substrate with an R group thereby creating a third generation substrate, the third generation substrate comprising the R group and at least one P_{SD} of the substrate; (vi) repeating steps (ii) and (iii) using the enzyme and substituting the substrate with the third generation substrate; identifying a third generation substrate having a relative k_{cat}/Kₘ ratio of not less than 2; wherein the third generation substrate has activity equivalent to at least one secondary specific determinant (S_{SD}); thereby synthesizing a compound having binding activity to the active site of an enzyme. In one preferred embodiment, the enzyme is selected from the group consisting of proteases, kinases, phosphatases, hydrolases, oxidoreductases, isomerases, transferases, methylases, acetylases, ligases, and lyases. In a more preferred embodiment, the substrate has a relative molecular mass (Mᵣ) of between about 150 daltons (Da) and about 500 Da. In a more preferred embodiment, the R group has a relative molecular mass of between about 175 Da and about 400 Da. In another more preferred embodiment, the substrate has a relative molecular mass of between about 200 Da and about 300 Da. In a more preferred embodiment, the R group has the properties of Mᵣ≦300, clogP≦5, hydrogen bond donor (HBD)≦5, and hydrogen bond acceptor (HBA)≦10. In another more preferred embodiment, the R group has the properties of Mᵣ<−300, clogP≦3, hydrogen bond donor (HBD)≦1, hydrogen bond acceptor (HBA)≦4, rotatable bonds ≦4, and at least one heteroaromatic or aromatic ring.

The invention also provides the use of a composition identified using the methods provided herein for the manufacture of a medicament for the treatment of a condition, a disease, or a disorder. In a preferred embodiment the condition, disease, or disorder is selected from the group consisting of neoplastic disorders such as, but not limited to, adenocarcinoma, leukemia, lymphoma, melanoma, myeloma, sarcoma, and teratocarcinoma, and particularly cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; neurological disorders such as akathesia, Alzheimer's disease, amnesia, amyotrophic lateral sclerosis, bipolar disorder, catatonia, cerebral neoplasms, dementia, depression, Down syndrome, tardive dyskinesia, dystonias, epilepsy, Huntington's disease, multiple sclerosis, neurofibromatosis, Parkinson's disease, paranoid psychoses, schizophrenia, and Tourette's disorder; angina, anaphylactic shock, arrhythmias, asthma, cardiovascular shock, Cushing's syndrome, hypertension, hypoglycemia, myocardial infarction, migraine, and pheochromocytoma; a cell proliferative disorder such as, but not limited to, actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia; and in disorders relating to infection or inflammation such as, but not limited to, acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, benign prostatic hyperplasia, bronchitis, Chediak-Higashi syndrome, cholecystitis, Crohn's disease, atopic dermatitis, dermatomyositis, diabetes mellitus, emphysema, erythroblastosis fetalis, erythema nodosum, atrophic gastritis, glomerulonephritis, Goodpasture's syndrome, gout, chronic granulomatous diseases, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polycystic ovary syndrome, polymyositis, psoriasis, Reiter's syndrome, rheumatoid arthritis, scleroderma, severe combined immunodeficiency disease (SCID), Sjogren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infection.

### BRIEF DESCRIPTION OF THE DRAWINGS

FIG. 1 illustrates a schematic of the synthesis of R-Arg-pNA molecules.

FIG. 2 illustrates a schematic of the synthesis of R-(4-Bz) molecules.

FIG. 3 illustrates an exemplary schematic description of the invention.

FIG. 4 illustrates an exemplary protocol for utilization of R″-Val-pNA library for finding new inhibitors for the enzyme human neutrophil elastase (HNE).

FIG. 5 illustrates an exemplary protocol for utilization of Ac-Lys-(ε-linker-biotin)-Thr-Pro-R″ library for finding new inhibitors for the enzyme cdk2-cyclin A.

### MODES FOR CARRYING OUT THE INVENTION

The embodiments disclosed in this document are illustrative and exemplary and are not meant to limit the invention. Other embodiments can be utilized and structural changes can be made without departing from the scope of the claims of the present invention.

As used herein and in the appended claims, the singular forms “a,” “an,” and “the” include plural reference unless the context clearly dictates otherwise. Thus, for example, a reference to “a residue” includes a plurality of such residues, and a reference to “a binding site” is a reference to one or more binding sites and equivalents thereof, and so forth.

As used herein, the term “enzyme” means a compound having activity that provides an environment that enables a chemical bond to be formed or to be broken between two or more atoms. The enzyme can be organic or inorganic. The enzyme can be in a biochemical pathway that is used in cellular anabolism, metabolism, and/or catabolism of a compound. The enzyme can be in a chemical pathway that is used in a chemical synthesis a compound or degradation of a compound.

The term “substrate” means a compound that can interact with an enzyme. The substrate can also bind to the enzyme reversibly or irreversibly. More than one substrate can be used with an enzyme.

The term “product” means a compound that is created due to the activity of the enzyme upon a substrate. More than one product can be created from one substrate.

The term “incubate” means a mixture of at least two compounds in an medium, wherein the medium can be a gas, liquid, gel, semi-solid, or solid phase, and wherein the compounds are combined for a period of time and at a particular temperature or a series of particular temperatures. The medium can be aqueous or can be non-aqueous. The medium can comprise a solvent.

The term “moiety” means a compound having a chemical formula that is recognized by those of skill in the art as having particular chemical and physical properties that are common to more than one similar compound. A moiety can include, but is not limited to, an amino acid residue, an oligopeptide, a synthetic peptide, a saccharide, a polysaccharide, a lipid, a phospholipid, a fatty acid, a glycoproterin, a proteoglycan, an aminoglycan, an alcohol amine, a nucleoside, a nucleotide, an oligonucleotide, a glycosyl phosphatidyl inositol, a steroid, and the like.

The invention disclosed herein provides methods of synthesizing synthetic substrates of enzymes that can be used to obtain inhibitors of the enzyme, both in vitro and in vivo. The invention provides a relatively inexpensive method of testing a plurality of compounds using the methods and materials disclosed herein to provide more effective inhibitors of enzymes that are useful as drugs for certain diseases, conditions, and disorders. The compounds can be combined with a suitable pharmaceutical carrier and used in the treatment of, for example, cancers such as colon cancer and pancreatic cancer, infectious diseases and disorders such as HIV and helminth infestation, and neurological disorders such as Alzheimer's Disease and Parkinson's Disease. The compound can be administered enterically as a digestible tablet, by injection in a suitable solvent solution, or placed as a pellet sub-dermally or within the cavity or confines of an organ.

### Target Enzymes

Enzymes that can be used to identify a ligand, a compound or substrate having binding activity, or a compound or substrate having inhibitory activity are well known in the art. A list of such enzymes can be found at the ExPASy (Expert Protein Analysis System) proteomics server of the Swiss Institute of Bioinformatics (SIB) website and database, and can be accessed via the world wide web at “isb-sib.ch”. The website and database identify enzymes, substrates, cofactors, and products that can be used with the instant invention to identify novel compounds having binding and/or inhibitory activity for the enzyme.

Preferred enzymes that are of particular use with the instant invention are disclosed herein.
Proteases
   a. serine proteases
   b. cysteine proteases
   c. aspartyl proteases
   d. metalloproteases
   e. threonine proteases
Protein kinases
   a. serine/threonine kinases
   b. tyrosine kinases
Protein phosphatases
Protein tyrosine phosphatases
Phospholipases
Hydrolases
   a. Fatty acid amide hydrolases
   b. Glycosidases
   c. Esterases
Proline hydroxylases
Oxidoreductases
   a. CYP450
   b. Monoxygenases
   c. dihydrofolatereductases
   d. alcohol dehydrogenases
Nitric oxide synthase
Deoxyribonucleases
Ribonucleases
ATPases
GTPases
Phosphodiesterases
Acetyltransferases
Ubiquitin-conjugating enzymes
Glycosyltransferases
Isomerases
Ligases

### Substrates and Ligands

The invention contemplates synthesis of and methods of use for substrates and ligands to identify novel inhibitors of enzymes and other ligand-binding molecules. The following table illustrates exemplary structures that can be so used (Table 1).

TABLE 1

| Target category | Target name | Endogenous substrate sequence | First substrate |
|---|---|---|---|
| Aspartic Protease | BACE | Glu-Val-Asn-Leu-Asp-Ala-Glu-Phe | R-Leu-Ala-Ala-Ala |
| | | | Ac-Val-Asn-Leu-R |
| Aspartic Protease | HIV-1* | Ser-Phe-Asn-Phe-Pro-Ile-Ser-Pro | R-Phe-Pro-Ile-Ser-Pro |
| | | | Ser-Phe-Asn-Phe-R |
| Aspartic Protease | Plasmepsin | Glu-Arg-Met-Phe-Leu-Ser-Phe-Pro | R-Phe-Leu-Ser-Phe-Pro |
| | | | Ac-Arg-Met-Phe-Leu-R |
| POP Peptidase | Depeptidyl peptidase IV | Gly-Pro-pNA | R-Pro-pNA |
| | (DPPIV)* | | |
| POP Peptidase | Fibroblast activation | Gly-Pro-pNA | R-Pro-pNA |
| | protein (FAP)* | | |
| POP Peptidase | Prolyl oligopeptidase | Z-Gly-Pro-pNA | R-Pro-pNA |
| | (POP)* | | |
| POP Peptidase | Dipeptidyl peptidase | Gly-Pro-pNA | R-P-pNA |
| | VII* | | |
| POP Peptidase | Oligopeptidase B | Z-Arg-Arg-AMC | R-Arg-AMC |
| Cysteine Protease | Caspase-1 | Tyr-Val-Ala-Asp-AMC | R-Asp-AMC |
| Cysteine Protease | Caspase-3 | Asp-Glu-Val-Asp-AMC | R-Asp-AMC |
| Cysteine Protease | Calpain | Gln-Glu-Val-Tyr-Gly-Met-Met-Pro | R-Tyr-pNA |
| Cysteine Protease | Cathepsin K | Val-Pro-Leu-Ser-His-Ser-Arg-Ser | R-Arg-pNA |
| Metalloprotease | MMP-9 | Gly-Phe-Gln-Gly-Ile-Phe-Gly-Gln | R-Ile-Phe-Gly-Gln |
| Metalloprotease | ADAM-17 (TACE) | Leu-Ala-Gln-Ala-Val-Arg-Ser-Ser | R-Val-Arg-Ser-Ser |
| | | | Leu-Ala-Gln-Ala-R |
| Metalloprotease | Aminopeptidase M | H-Met-pNA | R-Met-pNA |
| Serine Protease | Factor Xa | Bz-Ile-Glu-Gly-Arg-pNA | R-Arg-pNA |
| Serine Protease | Urokinase | Tos-Gly-Pro-Arg-pNA | R-Arg-pNA |
| Serine Protease | Neutrophil elastase | MeOSuc-Ala-Ala-Pro-Val-pNA | R-Val-pNA |
| Deacetylase | Histone deacetylase | Ac-Leu-Gly-Lys(Ac)-AMC | R-Lys(Ac)-AMC |
| Phospholipid-dependent | Akt (PK-B) | Arg-Thr-Asp-Ser-Tyr-Ser-Ala-Gly | R-Ser-Tyr, R-Ser-Phe |
| Kinase | | | Ac-Ser-Tyr-R, Ac-Ser- |
| | | | Phe-R |
| CMGC Kinase | GSK3b* | Pro-Pro-Ser-Pro-Ser-Leu-(p)Ser | R-Ser-Pro |
| | | | Ac-Ser-Pro-R |
| CMGC Kinase | P38* | Pro-Leu-Thr-Pro-Glu-Ser-Pro | R-Ser-Pro |
| | | | Ac-Ser-Pro-R |
| CMGC Kinases | JNK1, JNK2*, JNK3 | Ala-Ala-Ser-Pro-Pro-Ala-Ala | R-Ser-Pro |
| | | | Ac-Ser-Pro-R |
| CMGC Kinases | Cdk2* and Cdk5* | Pro-Phe-Ser-Pro-Ser-Gln-Phe | R-Ser-Pro |
| | | | Ac-Ser-Pro-R |
| CMGC Kinase | mTOR* | Ser-Thr-Thr-Pro-Gly-Gly-Thr | R-Thr-Pro |
| | | | Ac-Thr-Pro-R |
| Hydroxylase | HIF-Pro-4-hydroxylase* | Leu-Glu-Met-Leu-Ala-Pro | R-Pro |
| Hydroxylase | Collagen pro-4- | Pro-Pro-Gly-Pro-Pro-Gly | R-Pro |
| | hydroxylase* | | R-Pro-Gly |
| Phosphatase | PTP1b | Lys-Asp-Asp-Glu-(p)Tyr-Asn-Pro-Ala | R-(p)Tyr, Ac-(p)Tyr-R |
| Tyrosine Kinase | p60Src kinase | Gly-Ile-Tyr-Trp-His-His-Tyr | Ac-Ile-Tyr-R, |
| | | | R-Ile-Tyr |
| Hydrolase | Fatty acid amide | arachidonylethanolamide | R-ethanolamide |
| | hydrolase | | |

*proline-directed
R: R-group
pNA: para-nitroanilide
Bz: 4-aminomethylbenzamidine
Boc: t-butoxycarbonyl
Fmoc: flurenylmethoxycarbonyl
MeOSuc: methoxysuccinyl
Ac: acetyl
AMC: amido-4-methylcoumarin
AFC: amido-4-trifluoromethylcoumarin
Z: benzyloxycarbonyl
(p)Tyr = phosphotyrosine
(p)Ser = phosphoserine

Substrates for many, if not most, enzymes are well known in the art. The substrates include, but are not limited to, substrates in base, nucleoside, and nucleotide synthesis, substrates for serine, threonine, aspartyl, cysteine and metallo-proteases, substrates of oxidoreductases, substrates of serine/threonine and tyrosine kinases, substrates of protein phosphatases, substrates of lipid kinases, substrates of adenylate cyclase and of guanylyl cyclase, substrates of phosphodiesterases, substrates of nitric oxide synthase, substrates of lipid metabolism, substrates of steroid metabolism, substrates of ATP-dependent transporters, ligands of receptor proteins, such as, but not limited to, steroid hormones, arachidonic acid derivatives such as eicosenoids, ethanolamides, thyroid hormones, small peptide hormones such as endorphins and GnRH, neurotransmitters such as chatecholamines, acetylcholine, 4-aminobutyrate, 5-hydroxytryptamine, glutamate, histamine, aspartate, antigens, domains involved in protein-protein interaction, such as PDZ domains, RDG domains, leucine zipper domains, insulin/ILR domains, MHC class I and MHC class II/TRC domains, EGF domains, plekstrin domains, domains involved in modified residue/protein interactions, such as SH2 and SH3 domains, and the like.

Analyses of enzyme activity are well known to those in the art. For example, activity of enzymes can be determined by measuring the rate of reaction of a substrate to produce a product or products. The product(s) can be measured using a number of analytic methods that indirectly or directly measure the product concentration in the incubate or sample, including, but not limited to, change in optical density of the incubate, colorimetry, fluorimetry, mass-spectroscopy, radioisotope analysis, pH analysis, phase partition of product, electrochemical analysis of product, or the like.

The rate of formation of a product can be related to the initial enzyme concentration(s), the initial substrate concentration(s), and concentrations of initial co-factors, buffers, and ions in the incubate or sample. The rate of enzyme reaction can be used to determine kinetic parameters of the enzyme-substrate-product reaction using methods well known to those in the art. Methods for analyzing enzymes can be found, for example, Methods in Enzymology (1955-, vol. 1-, Academic Press (Elsevier), Burlington Mass.) and “A Study of Enzymes” Volume I and Volume II (Stephen A. Kuby, editor, (1990) CRC Press, Taylor & Francis, Baton Rouge Fla.).

The underlying concept and advantage of the invention is that substrate turnover requires the substrate to have precise binding in the enzyme's active site. Hence detection of the product formation by the enzyme is between 100 and 100 times more sensitive than established means that measure the percentage inhibition of the enzyme. Another advantage of the invention is that the same library of substrates can be used for different enzymes, for example comparisons between kallikreins and thrombin/coagulation pathway proteases or between different CMGC kinases.

### Reaction Conditions

Conditions for incubating enzymes with substrates and inhibitors are well known to those of skill in the art and can be found in the relevant literature, as disclosed above. The incubate can comprise salts, buffers, detergents, metal ions, chelating agents, co-factors, thiol-group compounds, phospholipids, cell extracts (including membranes), and the like.

The incubate can be incubated at a temperature at which optimal binding of the substrate or compound to the enzyme. The temperature can be derived empirically, using methods well known in the art. For example, the incubate mixture can be incubated at temperature from about 4° C. to about 95° C., from about 8° C. to about 80° C., from about 10° C. to about 60° C., from about 15° C. to about 45° C., or from about 16° C. to about 25° C. The temperature can be about, for example, 4° C., 6° C., 8° C., 10° C., 12° C., 16° C., 18° C., 20° C., 22° C., 24° C., 26° C., 28° C., 30° C., 33° C., 37° C., 40° C., 42° C., 45° C., 48° C., 50° C., 55° C., 57° C., 60° C., 65° C., 70° C., 75° C., 80° C., 85° C., 90° C., or 95° C.

Further, the substrate and enzyme in the incubate can be incubated together for a set time to produce sufficient detectable product. Compound can, optionally, be included in the incubate. In general, the set time can be predetermined using substrate and enzyme mixtures. The set time can vary from between seconds to hours, or even days. Set times for incubations are well known to those of skill in the art and can be found in the relevant literature, as disclosed above. For example, the set time can be about two seconds, about five seconds, about ten seconds, about twenty seconds, about thirty, about forty, about fifty, about sixty seconds, about ninety seconds, about two minutes, about three minutes, about four minutes, about five minutes, about ten minutes, about fifteen minutes, about thirty minutes, about forty five minutes, about sixty minutes, about ninety minutes, about two hours, about three hours, about four hours, bout five hours, about eight hours, about ten hours, about twelve hours, about fifteen hours, about eighteen hours, about twenty hours, about twenty four hours, about thirty hours, about thirty six hours, about forty hours, and about fifty hours. Longer times may be necessary to produce sufficient detectable product when small quantities of or relatively inactive enzymes are used with the instant invention.

### Determination of Kinetic Parameters

The kinetics of enzyme reactions that identify characteristics of an enzyme-substrate reaction, such as substrate specificity, substrate inhibition, product inhibition, rate of reaction, catalytic constant, and the like can be used to identify those reaction samples comprising a substrate of interest. In addition, when determining such characteristics, many variable factors must be taken into account, including, but not limited to, pH, temperature of the reaction, substrate (and co-factor, if relevant) concentration, concentration of any mono- or polyvalent ion co-factors, purity of enzyme, and enzyme concentration. Cornish-Bowden (1979 and 2000) provides an excellent resource as a review and methods for identifying such characteristics (Cornish-Bowden, A. (1979) Fundamentals of Enzyme Kinetics, Butterworth-Heinemann, London; Ibid (2000) third edition, Portland Press, UK). (See particularly, Cornish-Bowden (1979), where chapters 2, 3, and 5 disclose and teach kinetic parameters.)

Particular parameters to measure and take into account are rate of reaction (v) and substrate concentration (s) from which maximum velocity V (also known as Vₘₐₓ) and the Michaelis constant K_{M} can be determined. Of use are plots of v against s, 1/v against 1/s, and s/v against s, from which V and K_{M} can be determined. “Substrate specificity” can be calculated from dividing V by K_{M}. “Catalytic constant” (also known as “turnover number”) k_{cat} is calculated by dividing V by e₀, where e₀ is the initial concentration of enzyme. “Specificity constant” is calculated by dividing k_{cat} by K_{M}.

In addition there are many software computer programs that can automate calculation of these kinetic parameters. Examples of such programs, include but are not limited to, MM: Michaelis-Menten Enzyme Kinetics Software (Department of Mathematics and Statistics, University of North Carolina, Wilmington N.C.); MICMEN (Dept. Bioengineering, University of Washington, Seattle Wash.); SIGMAPLOT (Systat Software Inc., San Jose Calif.); NONMEM software (UCSF, San Francisco Calif. and GloboMax, LLC, Hanover, Md.); VISUALENZYMICS models (SoftZymics, Inc. Princeton N.J.).

Conditions and Disorders Associated with Enzyme Activity

As noted herein, many diseases, conditions, and disorders have been associated with alterations in expression of genes encoding enzymes or in the activity of enzymes, ion channels, ligand-binding proteins, transmembrane proteins, nuclear receptors, and regulatory proteins. These include, but are not limited to, neoplastic disorders such as adenocarcinoma, leukemia, lymphoma, melanoma, myeloma, sarcoma, and teratocarcinoma, and particularly cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; neurological disorders such as akathesia, Alzheimer's disease, amnesia, amyotrophic lateral, sclerosis, bipolar disorder, catatonia, cerebral neoplasms, dementia, depression, Down syndrome, tardive dyskinesia, dystonias, epilepsy, Huntington's disease, multiple sclerosis, neurofibromatosis, Parkinson's disease, paranoid psychoses, schizophrenia, and Tourette's disorder; angina, anaphylactic shock, arrhythmias, asthma, cardiovascular shock, Cushing's syndrome, hypertension, hypoglycemia, myocardial infarction, migraine, and pheochromocytoma; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia; and in disorders relating to infection or inflammation such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, benign prostatic hyperplasia, bronchitis, Chediak-Higashi syndrome, cholecystitis, Crohn's disease, atopic dermatitis, dermatomyositis, diabetes mellitus, emphysema, erythroblastosis fetalis, erythema nodosum, atrophic gastritis, glomerulonephritis, Goodpasture's syndrome, gout, chronic granulomatous diseases, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polycystic ovary syndrome, polymyositis, psoriasis, Reiter's syndrome, rheumatoid arthritis, scleroderma, severe combined immunodeficiency disease (SCID), Sjogren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infection.

### Definitions and Conventions

The definitions and explanations below are for the terms as used throughout this entire document including both the specification and the claims.

Conventions for Formulas and Definitions of Variables

TLC refers to thin-layer chromatography.

HPLC refers to high pressure liquid chromatography.

THF refers to tetrahydrofuran.

DMF refers to dimethylformamide.

EDC refers to ethyl-1-(3-dimethylaminopropyl)carbodiimide or 1-(3-dimethylaminopropyl)-3-ethylcarbodiimide hydrochloride.

HOBt refers to 1-hydroxy benzotriazole hydrate.

NMM refers to N-methylmorpholine.

NBS refers to N-bromosuccinimide.

TEA refers to triethylamine.

BOC refers to 1,1-dimethylethoxy carbonyl or t-butoxycarbonyl, represented schematically as —CO—O—C(CH₃)₃.

CBZ refers to benzyloxycarbonyl, —CO—O—CH₂-phenyl

FMOC refers to 9-fluorenylmethyl carbonate.

TFA refers to trifluoracetic acid, CF₃—COOH.

CDI refers to 1,1′-carbonyldiimidazole.

When solvent pairs are used, the ratios of solvents used are volume/volume (v/v).

When the solubility of a solid in a solvent is used the ratio of the solid to the solvent is weight/volume (wt/v).

BOP refers to benzotriazol-1-yloxy-tris(dimethylamino)phosphonium hexafluorophosphate.

TBDMSCI refers to t-butyldimethylsilyl chloride.

TBDMSOTf refers to t-butyldimethylsilyl trifluosulfonic acid ester.

By “alkyl” and “IC₁-C₆ alkyl” in the invention is meant straight or branched chain alkyl groups having 1-6 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. It is understood that in cases where an alkyl chain of a substituent (for example, of an alkyl, alkoxy or alkenyl group) is shorter or longer than 6 carbons, it will be so indicated in the second “C” as, for example, “C₁-C₁₀” indicates a maximum of 10 carbons.

By “alkoxy” and “C₁-C₆ alkoxy” in the invention is meant straight or branched chain alkyl groups having 1-6 carbon atoms, attached through at least one divalent oxygen atom, such as, for example, methoxy, ethoxy, propoxy, isopropoxy, n-butoxy, sec-butoxy, tert-butoxy, pentoxy, isopentoxy, neopentoxy, hexoxy, and 3-methylpentoxy.

By the term “halogen” in the invention is meant fluorine, bromine, chlorine, and iodine.

“Alkenyl” and “C₂-C₆ alkenyl” means straight and branched hydrocarbon radicals having from 2 to 6 carbon atoms and from one to three double bonds and includes, for example, ethenyl, propenyl, 1-but-3-enyl, 1-pent-3-enyl, 1-hex-5-enyl and the like.

“Alkynyl” and “C₂-C₆ alkynyl” means straight and branched hydrocarbon radicals having from 2 to 6 carbon atoms and one or two triple bonds and includes ethynyl, propynyl, butynyl, pentyn-2-yl and the like.

As used herein, the term “cycloalkyl” refers to saturated carbocyclic radicals having three to twelve carbon atoms. The cycloalkyl can be monocyclic, or a polycyclic fused system. Examples of such radicals include cyclopropyl, cyclobutyl, cyclopentyl, cyclohexyl and cycloheptyl. The cycloalkyl groups herein are unsubstituted or, as specified, substituted in one or more substitutable positions with various groups. For example, such cycloalkyl groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆haloalkoxy, amino(C₁-C₆)alkyl, mono(C₁-C₆)alkylamino(C₁-C₆)alkyl or di(C₁-C₆) alkylamino (C₁-C₆) alkyl.

By “aryl” is meant an aromatic carbocyclic group having a single ring (for example, phenyl), multiple rings (for example, biphenyl), or multiple condensed rings in which at least one is aromatic, (for example, 1,2,3,4-tetrahydronaphthyl, naphthyl), which is optionally mono-, di-, or trisubstituted. Preferred aryl groups of the invention are phenyl, 1-naphthyl, 2-naphthyl, indanyl, indenyl, dihydronaphthyl, tetralinyl or 6,7,8,9-tetrahydro-5H-benzo[a]cycloheptenyl. The aryl groups herein are unsubstituted or, as specified, substituted in one or more substitutable positions with various groups. For example, such aryl groups may be optionally substituted with, for example, C₁-C₆alkyl, C₁-C₆alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆haloalkyl, C₁-C₆haloalkoxy, amino(C₁-C₆)alkyl, mono(C₁-C₆)alkylamino(C₁-C₆)alkyl, di(C₁-C₆)alkylamino(C₁-C₆)alkyl, —COOH, —C(═O)O(C₁-C₆alkyl), —C(═O)NH₂, —C(═O)N(mono- or di-C₁-C₆alkyl), —S(C₁-C₆alkyl), —SO₂(C₁-C₆alkyl), —O—C(═O) (C₁-C₆alkyl), —NH—C(═O)—(C₁-C₆alkyl), —N(C₁-C₆alkyl)-C(═O)—(C₁-C₆alkyl), —NH—SO₂—(C₁-C₆alkyl), —N(C₁-C₆alkyl)-SO₂—(C₁-C₆alkyl), —NH—C(═O)NH₂, —NH—C(═O)N(mono- or di-C₁-C₆ alkyl), —NH(C₁-C₆alkyl)-C(═O)—NH₂ or —NH(C₁-C₆alkyl)-C(═O)—N-(mono- or di-C₁-C₆alkyl).

By “heteroaryl” is meant one or more aromatic ring systems of 5-, 6-, or 7-membered rings which includes fused ring systems of 9-11 atoms containing at least one and up to four heteroatoms selected from nitrogen, oxygen, or sulfur. Preferred heteroaryl groups of the invention include pyridinyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocumarinyl, chromonyl, chromanonyl, pyridinyl-N-oxide, tetrahydroquinolinyl, dihydroquinolinyl, dihydroquinolinonyl, dihydroisoquinolinonyl, dihydrocumarinyl, dihydroisocumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. The heteroaryl groups herein are unsubstituted or, as specified, substituted in one or more substitutable positions with various groups. For example, such heteroaryl groups may be optionally substituted with C₁-C₆ alkyl, C₁-C₆alkoxy, halogen, hydroxy, cyano, nitro, amino, mono (C₁-C₆) alkylamino, di(C₁-C₆) alkylamino, C₂-C₆alkenyl, C₂-C₆alkynyl, C₁-C₆haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl, —COOH, —C(═O)O(C₁-C₆alkyl), —C(═O)NH₂, —C(═O)N(mono- or di-C₁-C₆alkyl), —S(C₁-C₆alkyl), —SO₂(C₁-C₆alkyl), —O—C(═O)(C₁-C₆ alkyl), —NH—C(═O)—(C₁-C₆alkyl), —N(C₁-C₆alkyl)-C(═O)—(C₁-C₆alkyl), —NH—SO₂—(C₁-C₆alkyl), —N(C₁-C₆ alkyl)-SO₂—(C₁-C₆alkyl), —NH—C(═O)NH₂, —NH—C(═O)N(mono- or di-C₁-C₆alkyl), —NH(C₁-C₆alkyl)-C(═O)—NH₂ or —NH(C₁-C₆alkyl)-C(═O)—N-(mono- or di-C₁-C₆alkyl).

By “heterocycle”, “heterocycloalkyl” or “heterocyclyl” is meant one or more carbocyclic ring systems of 3-, 4-, 5-, 6-, or 7-membered rings which includes fused ring systems of 9-11 atoms containing at least one and up to four heteroatoms selected from nitrogen, oxygen, or sulfur. Preferred heterocycles of the invention include morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, azepanyl, diazepanyl, tetrahydrothienyl S-oxide, tetrahydrothienyl S,S-dioxide and homothiomorpholinyl S-oxide. The heterocycle groups herein maybe unsubstituted or, as specified, substituted in one or more substitutable positions with various groups. For example, such heterocycle groups may be optionally substituted with C₁-C₆alkyl, C₁-C₆alkoxy, halogen, hydroxy, cyano, nitro, amino, mono(C₁-C₆) alkylamino, di(C₁-C₆) alkylamino, C₂-C₆alkenyl, C₂-C₆alkynyl, C₁-C₆haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or (C═O).

The chemical formulas representing various compounds or molecular fragments in the specification and claims may contain variable substituents in addition to expressly defined structural features. These variable substituents are identified by a letter or a letter followed by a numerical subscript, for example, “Zᵢ” or “Rᵢ” where “i” is an integer. These variable substituents are either monovalent or bivalent, that is, they represent a group attached to the formula by one or two chemical bonds. For example, a group Z₁ would represent a bivalent variable if attached to the formula CH₃—C(=Z₁)H. Groups Rᵢ and Rⱼ would represent monovalent variable substituents if attached to the formula CH₃—CH₂—C(Rᵢ)(Rⱼ)H₂. When chemical formulas are drawn in a linear fashion, such as those above, variable substituents contained in parentheses are bonded to the atom immediately to the left of the variable substituent enclosed in parenthesis. When two or more consecutive variable substituents are enclosed in parentheses, each of the consecutive variable substituents is bonded to the immediately preceding atom to the left which is not enclosed in parentheses. Thus, in the formula above, both Rᵢ and Rⱼ are bonded to the preceding carbon atom. Also, for any molecule with an established system of carbon atom numbering, such as steroids, these carbon atoms are designated as Cᵢ, where “i” is the integer corresponding to the carbon atom number. For example, C₆ represents the 6 position or carbon atom number in the steroid nucleus as traditionally designated by those skilled in the art of steroid chemistry. Likewise the term “R₆” represents a variable substituent (either monovalent or bivalent) at the C₆ position.

Chemical formulas or portions thereof drawn in a linear fashion represent atoms in a linear chain. The symbol “—” in general represents a bond between two atoms in the chain. Thus CH₃—O—CH₂—CH(Rᵢ)—CH₃ represents a 2-substituted-1-methoxypropane compound. In a similar fashion, the symbol “=” represents a double bond, for example, CH₂═C(Rᵢ)—O—CH₃, and the symbol “≡” represents a triple bond, for example, HC≡C—CH(Rᵢ)—CH₂—CH₃. Carbonyl groups are represented in either one of two ways: —CO— or —C(═O)—, with the former being preferred for simplicity.

Chemical formulas of cyclic (ring) compounds or molecular fragments can be represented in a linear fashion. Thus, the compound 4-chloro-2-methylpyridine can be represented in linear fashion by N*═C(CH₃)—CH═CCl—CH═C*H with the convention that the atoms marked with an asterisk (*) are bonded to each other resulting in the formation of a ring. Likewise, the cyclic molecular fragment, 4-(ethyl)-1-piperazinyl can be represented by —N*—(CH₂)₂—N(C₂H₅)—CH₂—C*H₂.

A rigid cyclic (ring) structure for any compounds herein defines an orientation with respect to the plane of the ring for substituents attached to each carbon atom of the rigid cyclic compound. For saturated compounds which have two substituents attached to a carbon atom which is part of a cyclic system, —C(X₁)(X₂)— the two substituents may be in either an axial or equatorial position relative to the ring and may change between axial/equatorial. However, the position of the two substituents relative to the ring and each other remains fixed. While either substituent at times may lie in the plane of the ring (equatorial) rather than above or below the plane (axial), one substituent is always above the other. In chemical structural formulas depicting such compounds, a substituent (X₁) which is “below” another substituent (X₂) will be identified as being in the alpha configuration and is identified by a broken, dashed or dotted line attachment to the carbon atom, i.e., by the symbol “ - - - ” or “ . . . ”. The corresponding substituent attached “above” (X₂) the other (X₁) is identified as being in the beta configuration and is indicated by an unbroken line attachment to the carbon atom.

When a variable substituent is bivalent, the valences may be taken together or separately or both in the definition of the variable. For example, a variable Rᵢ attached to a carbon atom as —C(═Rᵢ)— might be bivalent and be defined as oxo or keto (thus forming a carbonyl group (—CO—) or as two separately attached monovalent variable substituents alpha-Rᵢ₋ⱼ and beta-Rᵢ₋ₖ. When a bivalent variable, Rᵢ, is defined to consist of two monovalent variable substituents, the convention used to define the bivalent variable is of the form “alpha-Rᵢ₋ⱼbeta-Rᵢ₋ₖ” or some variant thereof. In such a case both alpha-Rᵢ₋ⱼ and beta-Rⱼ₋ₖ are attached to the carbon atom to give —C(alpha-Rᵢ₋ⱼ)(beta-Rᵢ₋ₖ)—. For example, when the bivalent variable R₆, —C(═R₆)— is defined to consist of two monovalent variable substituents, the two monovalent variable substituents are alpha-R₆₋₁: beta-R₆₋₂, . . . alpha-R₆₋₉:beta-R₆₋₁₀, etc, giving —C(alpha-R₆₋₁)(beta-R₆₋₂)—, . . . —C(alpha-R₆₋₉)(beta-R₆₋₁₀)—, etc. Likewise, for the bivalent variable R₁₁, —C(═R₁₁)—, two monovalent variable substituents are alpha-R₁₁₋₁:beta-R₁₁₋₂. For a ring substituent for which separate alpha and beta orientations do not exist (for example, due to the presence of a carbon carbon double bond in the ring), and for a substituent bonded to a carbon atom which is not part of a ring the above convention is still used, but the alpha and beta designations are omitted.

Just as a bivalent variable may be defined as two separate monovalent variable substituents, two separate monovalent variable substituents may be defined to be taken together to form a bivalent variable. For example, in the formula —C₁(Rᵢ)H—C₂(Rⱼ)H—(C₁ and C₂ define arbitrarily a first and second carbon atom, respectively) Rᵢ and Rⱼ may be defined to be taken together to form (1) a second bond between C₁ and C₂ or (2) a bivalent group such as oxa (—O—) and the formula thereby describes an epoxide. When Rᵢ and Rⱼ are taken together to form a more complex entity, such as the group —X—Y—, then the orientation of the entity is such that C₁ in the above formula is bonded to X and C₂ is bonded to Y. Thus, by convention the designation “ . . . Rᵢ and Rⱼ are taken together to form —CH₂—CH₂—O—CO— . . . ” means a lactone in which the carbonyl is bonded to C₂. However, when designated “ . . . Rⱼ and Rᵢ are taken together to form —CO—O—CH₂—CH₂— the convention means a lactone in which the carbonyl is bonded to C₁.

The carbon atom content of variable substituents is indicated in one of two ways. The first method uses a prefix to the entire name of the variable such as “C₁-C₄”, where both “1” and “4” are integers representing the minimum and maximum number of carbon atoms in the variable. The prefix is separated from the variable by a space. For example, “C₁-C₄alkyl” represents alkyl of 1 through 4 carbon atoms, (including isomeric forms thereof unless an express indication to the contrary is given). Whenever this single prefix is given, the prefix indicates the entire carbon atom content of the variable being defined. Thus C₂-C₄ alkoxycarbonyl describes a group CH₃—(CH₂)ₙ—O—CO— where n is zero, one or two. By the second method the carbon atom content of only each portion of the definition is indicated separately by enclosing the “Cᵢ-Cⱼ” designation in parentheses and placing it immediately (no intervening space) before the portion of the definition being defined. By this optional convention (C₁-C₃)alkoxycarbonyl has the same meaning as C₁-C₄alkoxy-carbonyl because the “C₁-C₃” refers only to the carbon atom content of the alkoxy group. Similarly while both C₁-C₆alkoxyalkyl and (C₁-C₃)alkoxy(C₁-C₃)alkyl define alkoxyalkyl groups containing from 2 to 6 carbon atoms, the two definitions differ since the former definition allows either the alkoxy or alkyl portion alone to contain 4 or 5 carbon atoms while the latter definition limits either of these groups to 3 carbon atoms.

The R-group can consist of molecules selected by the following criteria. An R-group can be a large molecule, greater than 5000 Da (5 kDa) in size. In the alternative, it can be smaller that 5 kDa, for example, about 4.5 kDa, about 4 kDa, about 3.5 kDa, about 3 kDa, about 2.5 kDa, about 2 kDa, about 1.5 kDa, about 1 kDa, about 950 Da, about 900 Da, about 850 Da, about 800 Da, about 750 Da, about 700 Da, about 650 Da, about 600 Da, about 550 Da, about 500 Da, about 450 Da, about 400 Da, about 375 Da, about 350 Da, about 325 Da, about 300 Da, about 275 Da, about 250 Da, about 225 Da, about 200 Da, about 190 Da, about 180 Da, about 170 Da, about 160 Da, and about 150 Da in size.

The R-group can have the following properties: logP<15.0, has hydrogen bond donors <15, and has hydrogen bond acceptors <30. Alternatively, the R group has logP<5.0, has hydrogen bond donors <10, and has hydrogen bond acceptors <20, In another alternative, the R group has logP<5.0, has hydrogen bond donors <5, and has hydrogen bond acceptors <10. For example, logP can be about 1, about 2, about 3, about 4, about 5, about 6, about 7, about 8, about 9, about 10, about 11, about 12, about 13, about 14, and about 15. In another example, the R-group can have 1, 2, 3, 4, 5, 6, 7, 8, 9, and 10 hydrogen bond donors. In another example, the R-group can have 1, 2, 3, 4, 5, 6, 7, 8, 9, 10, 11, 12, 13, 14, 15, 16, 17, 18, 19, and 20 hydrogen bond acceptors.

The method can also be used to synthesize compounds having the general formulae:

R-X-AA3-(AAx)n-AA4-S (I)

S-AA3-(AAx)n-AA4-CO—R (II)

AA3-R-AA4-S (III)

wherein R(R-group) is H or is:
(I) R_{N-1} wherein R_{N-1} is selected from the group consisting of:
   (A) R_{N-aryl} wherein R_{N-aryl} is phenyl, 1-naphthyl, 2-naphthyl, tetralinyl, indanyl, or 6,7,8,9-tetrahydro-5H-benzo[a]cycloheptenyl, optionally substituted with one, two or three of the following substituents which can be the same or different and are:
      (1) C₁-C₆ alkyl, optionally substituted with one, two or three substituents selected from the group consisting of C₁-C₃ alkyl, —F, —Cl, —Br, —I, —OH, —SH, —C≡N, —CF₃, C₁-C₃ alkoxy, —NR₁₋ₐR_{1-b} wherein R₁₋ₐ and R_{1-b} are independently —H or C₁-C₆ alkyl,
      (2) —OH,
      (3) —NO₂,
      (4) —F, —Cl, —Br, —I,
      (5) —CO—OH,
      (6) —C≡N,
      (7) —(CH₂)₀₋₄—CO—NR_{N-2}R_{N-3} wherein R_{N-2} and R_{N-3} are the same or different and are selected from the group consisting of:
         (a) —H,
         (b) —C₁-C₆ alkyl optionally substituted with one substituent selected from the group consisting of:
         (i) —OH,
         (ii) —NH₂,
         (c) —C₁-C₆ alkyl optionally substituted with one to three —F, —Cl, —Br, —I,
         (d) —C₃-C₇ cycloalkyl,
         (e) —(C₁-C₂ alkyl)-(C₃-C₇ cycloalkyl),
         (f) —(C₁-C₆ alkyl)-O—(C₁-C₃ alkyl),
         (g) —C₂-C₆ alkenyl with one or two double bonds,
         (h) —C₂-C₆ alkynyl with one or two triple bonds,
         (i) —C₁-C₆ alkyl chain with one double bond and one triple bond,
         (j) —R_{1-aryl} wherein R_{1-aryl} carries the same definition as R_{N-aryl}, which is defined above,
         (k) —R_{1-heteroaryl} wherein R_{1-heteroaryl} carries the same definition as R_{N-heteroaryl}, which is defined below,
      (8) —(CH₂)₀₋₄—CO—(C₁-C₁₂ alkyl),
      (9) —(CH₂)₀₋₄—CO—(C₂-C₁₂ alkenyl with one, two or three double bonds),
      (10) —(CH₂)₀₋₄—CO—(C₂-C₁₂ alkynyl with one, two or three triple bonds),
      (11) —(CH₂)₀₋₄—CO—(C₃-C₇ cycloalkyl),
      (12) —(CH₂)₀₋₄—CO—R_{1-aryl} wherein R_{1-aryl} is as defined above,
      (13) —(CH₂)₀₋₄—CO—R_{1-heteroaryl} wherein R₁-heteroaryl is as defined above,
      (14) —(CH₂)₀₋₄—CO—R_{1-heterocycle} wherein R_{1-heterocycle} is selected from the group consisting of:
         (a) morpholinyl,
         (b) thiomorpholinyl,
         (c) thiomorpholinyl s-oxide,
         (d) thiomorpholinyl s,s-dioxide,
         (e) piperazinyl,
         (f) homopiperazinyl,
         (g) pyrrolidinyl,
         (h) pyrrolinyl,
         (i) tetrahydropyranyl,
         (j) piperidinyl,
         (k) tetrahydrofuranyl,
         (l) tetrahythiophenyl,
         (m) homopiperidinyl,
         (n) imidazolidine,
         (o) imidazolidine dione, and
         (p) dithiane,
wherein the R_{1-heterocycle} group is bonded by any atom of the parent R_{1-heterocycle} group substituted by hydrogen such that the new bond to the R_{1-heteroaryl} group replaces the hydrogen atom and its bond, wherein heterocycle is optionally substituted with one thru four:
   (1) C₁-C₆ alkyl optionally substituted with one, two or three substituents selected from the group consisting of C₁-C₃ alkyl, —F, —Cl, —Br, —I, —OH, —SH, and —NR₁₋ₐR_{1-b} wherein R₁₋ₐ and R_{1-b} are as defined above, —C≡N, —CF₃, C₁-C₃ alkoxy,
   (2) C₂-C₆ alkenyl with one or two double bonds,
   (3) C₂-C₆ alkynyl with one or two triple bonds,
   (4) —F, Cl, —Br and —I,
   (5) C₁-C₆ alkoxy,
   (6) —O—C₁-C₆ alkyl optionally substituted with one thru three —F, —Cl, —Br, —I,
   (7) —NR_{N-2}R_{N-3} wherein R_{N-2} and R_{N-3} are as defined above,
   (8) —OH,
   (9) —C≡N,
   (10) C₃-C₇ cycloalkyl,
   (11) —CO—(C₁-C₄ alkyl),
   (12) —SO₂—NR₁₋ₐR_{1-b} wherein R₁₋ₐ and R_{1-b} are as defined above,
   (13) —CO—NR₁₋ₐR_{1-b} wherein R₁₋ₐ and R_{1-b} are as defined above,
   (14) —SO₂—(C₁-C₄ alkyl), or
   (15) ═O, with the proviso that when n₁ is zero R_{1-heterocycle} is not bonded to the carbon chain by nitrogen;
(15) —(CH₂)₀₋₄—CO—R_{N-4} wherein R_{N-4} is selected from the group consisting of morpholinyl, thiomorpholinyl, piperazinyl, piperidinyl, homomorpholinyl, homothiomorpholinyl, homomorpholinyl S-oxide, homothiomorpholinyl S,S-dioxide, pyrrolinyl and pyrrolidinyl wherein each group is optionally substituted with one, two, three, or four of: C₁-C₆ alkyl, (16) —(CH₂)₀₋₄—CO—O—R_{N-5} wherein R_{N-5} is selected from the group consisting of:
   (a) C₁-C₆ alkyl,
   (b) —(CH₂)₀₋₂—(R_{1-aryl}) wherein R_{1-aryl} is as defined above,
   (c) C₂-C₆ alkenyl containing one or two double bonds,
   (d) C₂-C₆ alkynyl containing one or two triple bonds,
   (e) C₃-C₇ cycloalkyl,
   (f) —(CH₂)₀₋₂—(R_{1-heteroaryl}) wherein R_{1-heteroaryl} is as defined above,
(17) —(CH₂)₀₋₄—SO₂—NR_{N-2}R_{N-3} wherein R_{N-2} and R_{N-3} are as defined above,
(18) —(CH₂)₀₋₄—SO—(C₁-C₈ alkyl),
(19) —(CH₂)₀₋₄—SO₂—(C₁-C₁₂ alkyl),
(20) —(CH₂)₀₋₄—SO₂—(C₃-C₇ cycloalkyl),
(21) —(CH₂)₀₋₄—N(H or R_{N-5})—CO—O—R_{N-5} wherein R_{N-5} can be the same or different and is as defined above,
(22) —(CH₂)₀₋₄—N(H or R_{N-5})—CO—N(R_{N-5})₂, wherein R_{N-5} can be the same or different and is as defined above,
(23) —(CH₂)₀₋₄—N—CS—N(R_{N-5})₂, wherein R_{N-5} can be the same or different and is as defined above,
(24) —(CH₂)₀₋₄—N(—H or R_{N-5})—CO—R_{N-2} wherein R_{N-5} and R_{N-2} can be the same or different and are as defined above,
(25) —(CH₂)₀₋₄—NR_{N-2}R_{N-3} wherein R_{N-2} and R_{N-3} can be the same or different and are as defined above,
(26) —(CH₂)₀₋₄—R_{N-4} wherein R_{N-4} is as defined above,
(27) —(CH₂)₀₋₄—O—CO—(C₁-C₆ alkyl),
(28) —(CH₂)₀₋₄—O—P(O)—(OR_{N-aryl-1})₂ wherein R_{N-aryl-1} is —H or C₁-C₄ alkyl,
(29) —(CH₂)₀₋₄—O—CO—N(R_{N-5})₂ wherein R_{N-5} is as defined above,
(30) —(CH₂)₀₋₄—O—CS—N(R_{N-5})₂ wherein R_{N-5} is as defined above,
(31) —(CH₂)₀₋₄-O—(R_{N-5})₂ wherein R_{N-5} is as defined above,
(32) —(CH₂)₀₋₄-O—(R_{N-5})₂—COOH wherein R_{N-5} is as defined above,
(33) —(CH₂)₀₋₄—S—(R_{N-5})₂ wherein R_{N-5} is as defined above,
(34) —(CH₂)₀₋₄-O—(C₁-C₆ alkyl optionally substituted with one, two, three, four, or five of: —F), —Cl, —Br, —I,
(35) C₃-C₇ cycloalkyl,
(36) C₂-C₆ alkenyl with one or two double bonds optionally substituted with C₁-C₃ alkyl, —F, —Cl, —Br, —I, —OH, —SH, —C≡N, —CF₃, C₁-C₃ alkoxy, —NR₁₋ₐR_{1-b} wherein R₁₋ₐ and R_{1-b} are as defined above,
   (37) C₂-C₆ alkynyl with one or two triple bonds optionally substituted with C₁-C₃ alkyl, —F, —Cl, —Br, —I, —OH, —SH, —C≡N, —CF₃, C₁-C₃ alkoxy, —NR₁₋ₐR_{1-b} wherein R₁₋ₐ and R_{1-b} are as defined above,
   (38) —(CH₂)₀₋₄—N(—H or R_{N-5})—SO₂—R_{N-2} wherein R_{N-5} and R_{N-2} can be the same of different and are as described above, or
   (39) —(CH₂)₀₋₄—C₃-C₇ cycloalkyl,
   (B) —R_{N-heteroaryl} wherein R_{N-heteroaryl} is selected from the group consisting of:
      (a) pyridinyl,
      (b) pyrimidinyl,
      (c) quinolinyl,
      (f) benzothienyl,
      (g) indolyl,
      (h) indolinyl,
      (i) pyridazinyl,
      (j) pyrazinyl,
      (k) isoindolyl,
      (l) isoquinolyl,
      (m) quinazolinyl,
      (n) quinoxalinyl,
      (o) phthalazinyl,
      (p) imidazolyl,
      (q) isoxazolyl,
      (r) pyrazolyl,
      (s) oxazolyl,
      (t) thiazolyl,
      (u) indolizinyl,
      (v) indazolyl,
      (w) benzothiazolyl,
      (x) benzimidazolyl,
      (y) benzofuranyl,
      (z) furanyl,
      (aa) thienyl,
      (bb) pyrrolyl,
      (cc) oxadiazolyl,
      (dd) thiadiazolyl,
      (ee) triazolyl,
      (ff) tetrazolyl,
      (ii) oxazolopyridinyl,
      (jj) imidazopyridinyl,
      (kk) isothiazolyl,
      (ll) naphthyridinyl,
      (mm) cinnolinyl,
      (nn) carbazolyl,
      (oo) β-carbolinyl,
      (pp) isochromanyl,
      (qq) chromanyl,
      (ss) tetrahydroisoquinolinyl,
      (tt) isoindolinyl,
      (uu) isobenzotetrahydrofuranyl,
      (vv) isobenzotetrahydrothienyl,
      (ww) isobenzothienyl,
      (xx) benzoxazolyl,
      (yy) pyridopyridinyl,
      (zz) benzotetrahydrofuranyl,
      (aaa) benzotetrahydrothienyl,
      (bbb) purinyl,
      (ccc) benzodioxolyl,
      (ddd) triazinyl,
      (eee) phenoxazinyl,
      (fff) phenothiazinyl,
      (ggg) pteridinyl,
      (hhh) benzothiazolyl,
      (iii) imidazopyridinyl,
      (jj) imidazothiazolyl,
      (kkk) dihydrobenzisoxazinyl,
      (lll) benzisoxazinyl,
      (mmm) benzoxazinyl,
      (nnn) dihydrobenzisothiazinyl,
      (ooo) benzopyranyl,
      (ppp) benzothiopyranyl,
      (qqq) coumarinyl,
      (rrr) isocumarinyl,
      (sss) chromonyl,
      (ttt) chromanonyl, and
      (uuu) pyridinyl-n-oxide,
wherein the R_{N-heteroaryl} group is bonded by any atom of the parent R_{N-heteroaryl} group substituted by hydrogen such that the new bond to the R_{N-heteroaryl} group replaces the hydrogen atom and its bond, wherein heteroaryl is optionally substituted with one, two, three, or four of:
   (1) C₁-C₆ alkyl, optionally substituted with one, two or three substituents selected from the group consisting of C₁-C₃ alkyl, —F, —Cl, —Br, —I, —OH, —SH, —C≡N, —CF₃, C₁-C₃ alkoxy, —NR₁₋ₐR_{1-b} wherein R₁₋ₐ and R_{1-b} are as defined above,
   (2) —OH,
   (3) —NO₂,
   (4) —F, —Cl, —Br, —I,
   (5) —CO—OH,
   (6) —C≡N,
   (7) —(CH₂)₀₋₄—CO—NR_{N-2}R_{N-3} wherein R_{N-2} and R_{N-3} are the same or different and are selected from the group consisting of:
      (a) —H,
      (b) —C₁-C₆ alkyl optionally substituted with one substitutent selected from the group consisting of:
         (i) —OH,
         (ii) —NH₂,
      (c) —C₁-C₆ alkyl optionally substituted with one to three —F, —Cl, —Br, —I,
      (d) —C₃-C₇ cycloalkyl,
      (e) —(C₁-C₂ alkyl)-(C₃-C₇ cycloalkyl),
      (f) —(C₁-C₆ alkyl)-O—(C₁-C₃ alkyl),
      (g) —C₂-C₆ alkenyl with one or two double bonds,
      (h) —C₂-C₆ alkynyl with one or two triple bonds,
      (i) —C₁-C₆ alkyl chain with one double bond and one triple bond,
      (j) —R_{1-aryl} wherein R₁-aryl, is as defined above, and
      (k)-R_{1-heteroaryl} wherein R_{1-heteroaryl} is as defined above,
   (8) —(CH₂)₀₋₄—CO—(C₁-C₁₂ alkyl),
   (9) —(CH₂)₀₋₄—CO—(C₂-C₁₂ alkenyl with one, two or three double bonds),
   (10) —(CH₂)₀₋₄—CO—(C₂-C₁₂ alkynyl with one, two or three triple bonds),
   (11) —(CH₂)₀₋₄—CO—(C₃-C₇ cycloalkyl),
   (12) —(CH₂)₀₋₄—CO—R_{1-aryl} wherein R_{1-aryl} is as defined above,
   (13) —(CH₂)₀₋₄-CO—R_{1-heteroaryl} wherein R_{1-heteroaryl} is as defined above,
   (14) —(CH₂)₀₋₄—CO—R_{1-heterocycle} wherein R_{1-heterocycle is as defined above,}
   (15) —(CH₂)₀₋₄—CO—R_{N-4} wherein R_{N-4} is selected from the group consisting of morpholinyl, thiomorpholinyl, piperazinyl, piperidinyl, homomorpholinyl, homothiomorpholinyl, homomorpholinyl S-oxide, homothiomorpholinyl S,S-dioxide, pyrrolinyl and pyrrolidinyl wherein each group is optionally substituted with one, two, three, or four of: C₁-C₆ alkyl,
   (16) —(CH₂)₀₋₄—CO—O—R_{N-5} wherein R_{N-5} is selected from the group consisting of:
      (a) C₁-C₆ alkyl,
      (b) —(CH₂)₀₋₂—(R_{1-aryl}) wherein R_{1-aryl} is as defined above,
      (c) C₂-C₆ alkenyl containing one or two double bonds,
      (d) C₂-C₆ alkynyl containing one or two triple bonds,
      (e) C₃-C₇ cycloalkyl, and
      (f) —(CH₂)₀₋₂—(R_{1-heteroaryl}) wherein R_{1-heteroaryl} is as defined above,
   (17) —(CH₂)₀₋₄—SO₂—NR_{N-2}R_{N-3} wherein R_{N-2} and R_{N-3} are as defined above,
   (18) —(CH₂)₀₋₄—SO—(C₁-C₈ alkyl),
   (19) —(CH₂)₀₋₄—SO₂—(C₁-C₁₂ alkyl),
   (20) —(CH₂)₀₋₄—SO₂—(C₃-C₇ cycloalkyl),
   (21) —(CH₂)₀₋₄—N(H or R_{N-5})—CO—O—R_{N-5} wherein R_{N-5} can be the same or different and is as defined above,
   (22) —(CH₂)₀₋₄—N(H or R_{N-5})—CO—N(R_{N-5})₂, wherein R_{N-5} can be the same or different and is as defined above,
   (23) —(CH₂)₀₋₄—N—CS—N(R_{N-5})₂, wherein R_{N-5} can be the same or different and is as defined above,
   (24) —(CH₂)₀₋₄—N(—H or R_{N-5})—CO—R_{N-2} wherein R_{N-5} and R_{N-2} can be the same or different and are as defined above,
   (25) —(CH₂)₀₋₄—NR_{N-2}R_{N-3} wherein R_{N-2} and R_{N-3} can be the same or different and are as defined above,
   (26) —(CH₂)₀₋₄—R_{N-4} wherein R_{N-4} is as defined above,
   (27) —(CH₂)₀₋₄—O—CO—(C₁-C₆ alkyl),
   (28) —(CH₂)₀₋₄—P(O)—(OR_{N-aryl-1})₂ wherein R_{N-aryl-1} is —H or C₁-C₄ alkyl,
   (29) —(CH₂)₀₋₄—O—CO—N(R_{N-5})₂ wherein R_{N-5} is as defined above,
   (30) —(CH₂)₀₋₄—O—CS—N(R_{N-5})₂ wherein R_{N-5} is as defined above,
   (31) —(CH₂)₀₋₄—O—(R_{N-5})₂ wherein R_{N-5} is as defined above,
   (32) —(CH₂)₀₋₄-O—(R_{N-5})₂—COOH wherein R_{N-5} is as defined above,
   (33) —(CH₂)₀₋₄-S—(R_{N-5})₂ wherein R_{N-5} is as defined above,
   (34) —(CH₂)₀₋₄-O—(C₁-C₆ alkyl optionally substituted with one, two, three, four, or five of: —F, —Cl, —Br, —I),
   (35) C₃-C₇ cycloalkyl,
   (36) C₂-C₆ alkenyl with one or two double bonds optionally substituted with C₁-C₃ alkyl, —F, —Cl, —Br, —I, —OH, —SH, —C N, —CF₃, C₁-C₃ alkoxy, —NR₁₋ₐR_{1-b} wherein R₁₋ₐ and R_{1-b} are as defined above,
   (37) C₂-C₆ alkynyl with one or two triple bonds optionally substituted with C₁-C₃ alkyl, —F, —Cl, —Br, —I, —OH, —SH, —C≡N, —CF₃, C₁-C₃ alkoxy, —NR₁₋ₐR_{1-b} wherein R₁₋ₐ and R_{1-b} are as defined above,
   (38) —(CH₂)₀₋₄—N(—H or R_{N-5})—SO₂—R_{N-2} wherein R_{N-5} and R_{N-2} can be the same of different and are as described above, or
   (39) —(CH₂)₀₋₄—C₃-C₇ cycloalkyl,
(C) R_{N-aryl}-W—R_{N-aryl},
(D) R_{N-aryl}-W—R_{N-heteroaryl},
(E) R_{N-aryl}-W-R_{N-1-heterocycle},
(F) R_{N-heteroaryl}—W-R_{N-aryl,}
(G) R_{N-heteroaryl}—W—R_{N-heteroaryl,}
(H) R_{N-heteroaryl}—W—R_{N-1-heterocycle},
(I) R_{N-heterocycle}—W—R_{N-aryl},
(J) R_{N-heterocycle}—W—R_{N-heteroaryl},
(K) R_{N-heterocycle}—W—R_{N-1-heterocycle},
   wherein W is
      (1) —(CH₂)₀₋₄—,
      (2) —O—,
      (3) —S(O)₀₋₂—,
      (4) —N(R_{N-5})— wherein R_{N-5} is as defined above, or
      (5) —CO—;
(II) —(C₁-C₁₀ alkyl) wherein alkyl is optionally substituted with one, two, or three substituents selected from the group consisting of:
   (A) —OH,
   (B) —C₁-C₆ alkoxy,
   (C) —C₁-C₆ thioalkoxy,
   (D) —CO—O—R_{N-8} wherein R_{N-8} is —H, C₁-C₆ alkyl or −φ,
   (E) —CO—NR_{N-2}R_{N-3} wherein R_{N-2} and R_{N-3} are the same or different and are as defined above,
   (F) —CO—R_{N-4} wherein R_{N-4} is as defined above,
   (G) —SO₂—(C₁-C₈ alkyl),
   (H) —SO₂—NR_{N-2}R_{N-3} wherein R_{N-2} and R_{N-3} are the same or different and are as defined above,
   (I) —NH—CO—(C₁-C₆ alkyl),
   (J) —NH—CO—O—R_{N-8} wherein R_{N-8} is as defined above,
   (K) —NR_{N-2}R_{N-3} wherein R_{N-2} and R_{N-3} are the same or different and are as defined above,
   (L) —R_{N-4} wherein R_{N-4} is as defined above,
   (M) —O—CO—(C₁-C₆ alkyl),
   (N) —O—CO—NR_{N-8}R_{N-8} wherein the R_{N-8}s are the same or different and are as defined above,
   (O) —O—(C₁-C₅ alkyl)-COOH,
   (P) —O—(C₁-C₆ alkyl optionally substituted with one, two, or three of: —F, —Cl, —Br, —I),
   (Q) —NH—SO₂—(C₁-C₆ alkyl), and
   (R) —F, —Cl,
(III) —X—O—(C₁-C₆ alkyl) wherein X is absent or is C₁-C₆ alkyl wherein alkyl is optionally substituted with one, two, or three substituents selected from the group consisting of:
   (A) —OH,
   (B) —C₁-C₆ alkoxy,
   (C) —C₁-C₆ thioalkoxy,
   (D) —CO—O—R_{N-8} wherein R_{N-8} is —H, C₁-C₆ alkyl or -.,
   (E) —CO—NR_{N-2}R_{N-3} wherein R_{N-2} and R_{N-3} are the same or different and are as defined above,
   (F) —CO—R_{N-4} wherein R_{N-4} is as defined above,
   (G) —SO₂—(C₁-C₈ alkyl),
   (H) —SO₂—NR_{N-2}R_{N-3} wherein R_{N-2} and R_{N-3} are the same or different and are as defined above,
   (I) —NH—CO—(C₁-C₆ alkyl),
   (J) —NH—CO—O—R_{N-8} wherein R_{N-8} is as defined above,
   (K) —NR_{N-2}R_{N-3} wherein R_{N-2} and R_{N-3} are the same or different and are as defined above,
   (L) —R_{N-4} wherein R_{N-4} is as defined above,
   (M) —O—CO—(C₁-C₆ alkyl),
   (N) —O—CO—NR_{N-8}R_{N-8} wherein the R_{N-8}s are the same or different and are as defined above,
   (O) —O—(C₁-C₅ alkyl)-COOH,
   (P) —O—(C₁-C₆ alkyl optionally substituted with one, two, or three of: —F, —Cl, —Br, —I),
   (Q) —NH—SO₂—(C₁-C₆ alkyl), and
   (R) —F, —Cl,
(IV) —(C₁-C₆ alkyl)-S—(C₁-C₆ alkyl) wherein alkyl is optionally substituted with one, two, or three substituents selected from the group consisting of:
   (A) —OH,
   (B) —C₁-C₆ alkoxy,
   (C) —C₁-C₆ thioalkoxy,
   (D) —CO—O—R_{N-8} wherein R_{N-8} is as defined above,
   (E) —CO—NR_{N-2}R_{N-3} wherein R_{N-2} and R_{N-3} are the same or different and are as defined above,
   (F) —CO—R_{N-4} wherein R_{N-4} is as defined above,
   (G) —SO₂—(C₁-C₈ alkyl),
   (H) —SO₂—NR_{N-2}R_{N-3} wherein R_{N-2} and R_{N-3} are the same or different and are as defined above,
   (I) —NH—CO—(C₁-C₆ alkyl),
   (J) —NH—CO—O—R_{N-8} wherein R_{N-8} is as defined above,
   (K) —NR_{N-2}R_{N-3} wherein R_{N-2} and R_{N-3} are the same or different and are as defined above,
   (L) —R_{N-4} wherein R_{N-4} is as defined above,
   (M) —O—CO—(C₁-C₆ alkyl),
   (N) —O—CO—NR_{N-8}R_{N-8} wherein the R_{N-8}s are the same or different and are as defined above,
   (O) —O—(C₁-C₅ alkyl)-COOH,
   (P) —O—(C₁-C₆ alkyl optionally substituted with one, two, or three of: —F, —Cl, —Br, —I),
   (Q) —NH—SO₂—(C₁-C₆ alkyl), and
   (R) —F, —Cl,
(V) —CH(—(CH₂)₀₋₂—O—R_{N-10})—(CH₂)₀₋₂—R_{N-aryl}/R_{N-heteroaryl}) wherein R_{N-aryl} and R_{N-heteroaryl} are as defined above, wherein R_{N-10} is selected from the group consisting of:
   (A) —H,
   (B) C₁-C₆ alkyl,
   (C) C₃-C₇ cycloalkyl,
   (D) C₂-C₆ alkenyl with one double bond,
   (E) C₂-C₆ alkynyl with one triple bond,
   (F) R_{1-aryl} wherein R_{1-aryl} is as defined above,
   (G) R_{N-heteroaryl} wherein R_{N-heteroaryl} is as defined above,
(VI) —(C₃-C₈ cycloalkyl) wherein alkyl is optionally substituted with one or two substituents selected from the group consisting of:
   (A) —(CH₂)₀₋₄—OH,
   (B) —(CH₂)₀₋₄—C₁-C₆ alkoxy,
   (C) —(CH₂)₀₋₄—C₁-C₆ thioalkoxy,
   (D) —(CH₂)₀₋₄—CO—O—R_{N-8} wherein R_{N-8} is —H, C₁-C₆ alkyl or -.,
   (E) —(CH₂)₀₋₄—CO—NR_{N-2}R_{N-3} wherein R_{N-2} and R_{N-3} are the same or different and are as defined above,
   (F) —(CH₂)₀₋₄—CO—R_{N-4} wherein R_{N-4} is as defined above,
   (G) —(CH₂)₀₋₄—SO₂—(C₁-C₈ alkyl),
   (H) —(CH₂)₀₋₄—SO₂—NR_{N-2}R_{N-3} wherein R_{N-2} and R_{N-3} are the same or different and are as defined above,
   (I) —(CH₂)₀₋₄—NH—CO—(C₁-C₆ alkyl),
   (J) —NH—CO—O—R_{N-8} wherein R_{N-8} is as defined above,
   (K) —(CH₂)₀₋₄—NR_{N-2}R_{N-3} wherein R_{N-2} and R_{N-3} are the same or different and are as defined above,
   (L) —(CH₂)₀₋₄—R_{N-4} wherein R_{N-4} is as defined above,
   (M) —O—CO—(C₁-C₆ alkyl),
   (N) —O—CO—NR_{N-8}R_{N-8} wherein the R_{N-8}s are the same or different and are as defined above,
   (O) —O—(C₁-C₅ alkyl)-COOH,
   (P) —O—(C₁-C₆ alkyl optionally substituted with one, two, or three of: —F, —Cl, —Br, —I),
   (Q) —NH—SO₂—(C₁-C₆ alkyl), and
   (R) —F, —Cl;
wherein R₂ and R₃ are the same or different and are:
   (I) C₁-C₆ alkyl, optionally substituted with one, two or three substituents selected from the group consisting of C₁-C₃ alkyl, —F, —Cl, —Br, —I, —OH, —SH, —C≡N, —CF₃, C₁-C₃ alkoxy, —COOH, —COO(C₁-C₆ alkyl), —CONR₁ₐR_{1b} and —NR₁₋ₐR_{1-b} wherein Rica and R_{1-b} are —H or C₁-C₆ alkyl,
   (II) —CH₂—S—(C₁-C₆ alkyl),
   (III) —CH₂—CH₂—S—(C₁-C₆ alkyl),
   (IV) C₂-C₆ alkenyl with one or two double bonds,
   (V) C₂-C₆ alkynyl with one or two triple bonds,
   (VI) —(CH₂)ₙ₁—(R_{1-aryl}) wherein n₁ is zero or one and wherein R_{1-aryl} is phenyl, 1-naphthyl, 2-naphthyl and indanyl, indenyl, dihydronaphthalyl, or tetralinyl each of which is optionally substituted with 1, 2, 3, or 4 of the following substituents on the aryl ring:
      (A) C₁-C₆ alkyl optionally substituted with one, two or three substituents selected from the group consisting of C₁-C₃ alkyl, —F, —Cl, —Br, —I, —OH, —SH, —NR₁₋ₐR_{1-b} wherein R₁₋ₐ and R_{1-b} are as defined above, —C≡N, —CF₃, C₁-C₃ alkoxy,
      (B) C₂-C₆ alkenyl with one or two double bonds,
      (C) C₂-C₆ alkynyl with one or two triple bonds,
      (D) —F, —Cl, —Br and —I,
      (E) C₁-C₆ alkoxy,
      (F) —O—C₁-C₆ alkyl optionally substituted with one thru three —F, or
      (G) —NR_{N-2}R_{N-3} wherein at each occurrence R_{N-2} and R_{N-3} are the same or different and are selected from the group consisting of:
         (a) —H,
         (b) —C₁-C₈ alkyl optionally substituted with one substituent selected from the group consisting of:
         (i) —OH,
         (ii) —NH₂,
         (iii) phenyl,
         (c) —C₁-C₈ alkyl optionally substituted with 1, 2, or 3 groups that are independently —F, —Cl, —Br, or —I,
         (d) —C₃-C₈ cycloalkyl,
         (e) —(C₁-C₂ alkyl)-(C₃-C₈ cycloalkyl),
         (f) —(C₁-C₆ alkyl)-O—(C₁-C₃ alkyl),
         (g) —C₂-C₆ alkenyl,
         (h) —C₂-C₆ alkynyl,
         (i) —C₁-C₆ alkyl chain with one double bond and one triple bond,
         (j) —R_{1-aryl},
         (k)-R_{1-heteroaryl},
         (l) —R_{1-heterocycle}, and
         (m) R_{N-2}, R_{N-3} and the nitrogen to which they are attached form a 5, 6, or 7 membered heterocycloalkyl or heteroaryl group, wherein said heterocycloalkyl or heteroaryl group is optionally fused to a benzene, pyridine, or pyrimidine ring, and said groups are unsubstituted or substituted with 1, 2, 3, 4, or 5 groups that at each occurrence are independently C₁-C₆ alkyl, C₁-C₆ alkoxy, halogen, halo C₁-C₆ alkyl, halo C₁-C₆ alkoxy, —CN, —NO₂, —NH₂, NH(C₁-C₆ alkyl), N(C₁-C₆ alkyl)(C₁-C₆ alkyl), —OH, —C(O)NH₂, —C(O)NH(C₁-C₆ alkyl), —C(O)N(C₁-C₆ alkyl)(C₁-C₆ alkyl), C₁-C₆ alkoxy C₁-C₆ alkyl, C₁-C₆ thioalkoxy, and C₁-C₆ thioalkoxy C₁-C₆ alkyl;
      (H) —OH,
      (I) —C≡N,
      (J) C₃-C₇ cycloalkyl,
      (K) —CO—(C₁-C₄ alkyl),
      (L) —SO₂—NR₁₋ₐR_{1-b} wherein Rica and R_{1-b} are as defined above,
      (M) —CO—NR₁₋ₐR_{1-b} wherein R₁₋ₐ and R_{1-b} are as defined above, or
      (N) —SO₂—(C₁-C₄ alkyl),
   (VII) —(CH₂)ₙ₁—(R_{1-heteroaryl}) wherein n₁ is as defined above and wherein R_{1-heteroaryl is selected from the group consisting of:}
      (A) pyridinyl,
      (B) pyrimidinyl,
      (C) quinolinyl,
      (F) benzothiophenyl,
      (G) indolyl,
      (H) indolinyl,
      (I) pyridazinyl,
      (J) pyrazinyl,
      (K) isoindolyl,
      (L) isoquinolyl,
      (M) quinazolinyl,
      (N) quinoxalinyl,
      (O) phthalazinyl,
      (P) imidazolyl,
      (Q) isoxazolyl,
      (R) pyrazolyl,
      (S) oxazolyl,
      (T) thiazolyl,
      (U) indolizinyl,
      (V) indazolyl,
      (W) benzothiazolyl,
      (X) benzimidazolyl,
      (Y) benzofuranyl,
      (Z) furanyl,
      (AA) thienyl,
      (BB) pyrrolyl,
      (CC) oxadiazolyl,
      (DD) thiadiazolyl,
      (EE) triazolyl,
      (FF) tetrazolyl,
      (GG) purinyl,
      (HH) 1,3-benzodioxolyl,
      (II) oxazolopyridinyl,
      (JJ) imidazopyridinyl,
      (KK) isothiazolyl,
      (ILL) naphthyridinyl,
      (MM) cinnolinyl,
      (NN) carbazolyl,
      (OO) β-carbolinyl,
      (PP) isochromanyl,
      (QQ) chromanyl,
      (RR) furazanyl,
      (SS) tetrahydroisoquinoline,
      (TT) isoindolinyl,
      (UU) isobenzotetrahydrofuranyl,
      (VV) isobenzotetrahydrothienyl,
      (WW) isobenzothiophenyl,
      (XX) benzoxazolyl,
      (YY) pyridopyridinyl,
      (ZZ) benzotetrahydrofuranyl, and
      (AAA) benzotetrahydrothienyl,
wherein the R_{1-heteroaryl} group is bonded to —(CH₂)ₙ₁— by any ring atom of the parent R_{N-heteroaryl} group substituted by hydrogen such that the new bond to the R_{1-heteroaryl} group replaces the hydrogen atom and its bond, wherein heteroaryl is optionally substituted with one thru four:
   (1) C₁-C₆ alkyl optionally substituted with one, two or three substituents selected from the group consisting of C₁-C₃ alkyl, —F, —Cl, —Br, —I, —OH, —SH, —NR₁₋ₐR_{1-b} wherein R₁₋ₐ and R_{1-b} are as defined above, —C≡N, —CF₃, C₁-C₃ alkoxy,
   (2) C₂-C₆ alkenyl with one or two double bonds,
   (3) C₂-C₆ alkynyl with one or two triple bonds,
   (4) —F, Cl, —Br and —I,
   (5) C₁-C₆ alkoxy,
   (6) —O—C₁-C₆ alkyl optionally substituted with one thru three —F,
   (7) —NR_{N-2}R_{N-3} wherein R_{N-2} and R_{N-3} are as defined above,
   (8) —OH,
   (9) —C≡N,
   (10) C₃-C₇ cycloalkyl,
   (11) —CO—(C₁-C₄ alkyl),
   (12) —SO₂—NR₁₋ₐR_{1-b} wherein R₁₋ₐ and R_{1-b} are as defined above,
   (13) —CO—NR₁₋ₐR_{1-b} wherein R₁₋ₐ and R_{1-b} are as defined above, or
   (14) —SO₂—(C₁-C₄ alkyl), with the proviso that when n₁ is zero R_{1-heteroaryl} is not bonded to the carbon chain by nitrogen;
(VIII) —(CH₂)ₙ₁—(R_{1-heterocycle}) wherein n₁ is as defined above and R_{1-heterocycle} is selected from the group consisting of:
   (A) morpholinyl,
   (B) thiomorpholinyl,
   (C) thiomorpholinyl S-oxide,
   (D) thiomorpholinyl S,S-dioxide,
   (E) piperazinyl,
   (F) homopiperazinyl,
   (G) pyrrolidinyl,
   (H) pyrrolinyl,
   (I) tetrahydropyranyl,
   (J) piperidinyl,
   (K) tetrahydrofuranyl,
   (L) tetrahythiophenyl,
   (M) homopiperidinyl,
   (N) imidazolidine,
   (O) imidazolidine dione, and
   (P) dithiane,
wherein the R_{1-heterocycle} group is bonded by any atom of the parent R_{1-heterocycle} group substituted by hydrogen such that the new bond to the R_{1-heteroaryl} group replaces the hydrogen atom and its bond, wherein heterocycle is optionally substituted with one thru four:
   (1) C₁-C₆ alkyl optionally substituted with one, two or three substituents selected from the group consisting of C₁-C₃ alkyl, —F, —Cl, —Br, —I, —OH, —SH, and —NR₁₋ₐR_{1-b} wherein R₁₋ₐ and R_{1-b} are as defined above, —C≡N, —CF₃, C₁-C₃ alkoxy,
   (2) C₂-C₆ alkenyl with one or two double bonds,
   (3) C₂-C₆ alkynyl with one or two triple bonds,
   (4) —F, Cl, —Br and —I,
   (5) C₁-C₆ alkoxy,
   (6) —O—C₁-C₆ alkyl optionally substituted with one thru three —F,
   (7) —NR_{N-2}R_{N-3} wherein R_{N-2} and R_{N-3} are as defined above,
   (8) —OH,
   (9) —C≡N,
   (10) C₃-C₇ cycloalkyl,
   (11) —CO—(C₁-C₄ alkyl),
   (12) —SO₂—NR₁₋ₐR_{1-b} wherein R₁₋ₐ and R_{1-b} are as defined above,
   (13) —CO—NR₁₋ₐR_{1-b} wherein R₁₋ₐ and R_{1-b} are as defined above,
   (14) —SO₂—(C₁-C₄ alkyl), or
   (15) ═O, with the proviso that when n₁ is zero R_{1-heterocycle} is not bonded to the carbon chain by nitrogen;
(IX) —(CH₂)₀₋₃—(C₃-C₇) cycloalkyl wherein cycloalkyl can be optionally substituted with one, two or three substituents selected from the group consisting of C₁-C₃ alkyl, —F, —Cl, —Br, —I, —OH, —SH, —C≡N, —CF₃, C₁-C₆ alkoxy, —O-phenyl, —CO—OH, —CO—O—(C₁-C₄ alkyl), and —NR₁₋ₐR_{1-b} wherein Rica and R_{1-b} are as defined above,
wherein R₄ is selected from the group consisting of:
   (I) —H,
   (II) C₁-C₆ alkyl, optionally substituted with one, two or three substituents selected from the group consisting of C₁-C₃ alkyl, —F, —Cl, —Br, —I, —OH, —SH, —C≡N, —CF₃, C₁-C₃ alkoxy, —NR₁₋ₐR_{1-b} wherein R₁₋ₐ and R_{1-b} are —H or C₁-C₆ alkyl,
   (III) —(CH₂)₀₋₄—R₂₋₁ wherein R₂₋₁ is R_{1-aryl} or R_{1-heteroaryl} wherein R_{1-aryl} is phenyl, 1-naphthyl, 2-naphthyl and indanyl, indenyl, dihydronaphthalyl, or tetralinyl each of which is optionally substituted with 1, 2, 3, or 4 of the following substituents on the aryl ring:
      (A) C₁-C₆ alkyl optionally substituted with one, two or three substituents selected from the group consisting of C₁-C₃ alkyl, —F, —Cl, —Br, —I, —OH, —SH, —NR₁₋ₐR_{1-b} wherein R₁₋ₐ, and R_{1-b} are as defined above, —C≡N, —CF₃, C₁-C₃ alkoxy,
      (B) C₂-C₆ alkenyl with one or two double bonds,
      (C) C₂-C₆ alkynyl with one or two triple bonds,
      (D) —F, —Cl, —Br and —I,
      (E) C₁-C₆ alkoxy,
      (F) —O—C₁-C₆ alkyl optionally substituted with one thru three —F,
      (G) —NR_{N-2}R_{N-3} wherein at each occurrence R_{N-2} and R_{N-3} are the same or different and are selected from the group consisting of:
         (a) —H,
         (b) —C₁-C₈ alkyl optionally substituted with one substituent selected from the group consisting of:
         (i) —OH,
         (ii) —NH₂,
         (iii) phenyl,
         (c) —C₁-C₈ alkyl optionally substituted with 1, 2, or 3 groups that are independently —F, —Cl, —Br, or —I,
         (d) —C₃-C₈ cycloalkyl,
         (e) —(C₁-C₂ alkyl)-(C₃-C₈ cycloalkyl),
         (f) —(C₁-C₆ alkyl)-O—(C₁-C₃ alkyl),
         (g) —C₂-C₆ alkenyl,
         (h) —C₂-C₆ alkynyl,
         (i) —C₁-C₆ alkyl chain with one double bond and one triple bond,
         (j) —R_{1-aryl},
         (k) —R_{1-heteroaryl},
         (l) —R_{1-heterocycle}, and
         (m) R_{N-2}, R_{N-3} and the nitrogen to which they are attached form a 5, 6, or 7 membered heterocycloalkyl or heteroaryl group, wherein said heterocycloalkyl or heteroaryl group is optionally fused to a benzene, pyridine, or pyrimidine ring, and said groups are unsubstituted or substituted with 1, 2, 3, 4, or 5 groups that at each occurrence are independently C₁-C₆ alkyl, C₁-C₆ alkoxy, halogen, halo C₁-C₆ alkyl, halo C₁-C₆ alkoxy, —CN, —NO₂, —NH₂, NH(C₁-C₆ alkyl), N(C₁-C₆ alkyl)(C₁-C₆ alkyl), —OH, —C(O)NH₂, —C(O)NH(C₁-C₆ alkyl), —C(O)N(C₁-C₆ alkyl)(C₁-C₆ alkyl), C₁-C₆ alkoxy C₁-C₆ alkyl, C₁-C₆ thioalkoxy, and C₁-C₆ thioalkoxy C₁-C₆ alkyl;
      (H) —OH,
      (I) —C≡N,
      (J) C₃-C₇ cycloalkyl,
      (K) —CO—(C₁-C₄ alkyl),
      (L) —SO₂—NR₁₋ₐR_{1-b} wherein R₁₋ₐ and R_{1-b} are as defined above,
      (M) —CO—NR₁₋ₐR_{1-b} wherein R₁₋ₐ and R_{1-b} are as defined above, or
      (N)—SO₂—(C₁-C₄ alkyl),
and R_{1-heteroaryl} is selected from the group consisting of:
   (A) pyridinyl,
   (B) pyrimidinyl,
   (C) quinolinyl,
   (F) benzothiophenyl,
   (G) indolyl,
   (H) indolinyl,
   (I) pyridazinyl,
   (J) pyrazinyl,
   (K) isoindolyl,
   (L) isoquinolyl,
   (M) quinazolinyl,
   (N) quinoxalinyl,
   (O) phthalazinyl,
   (P) imidazolyl,
   (Q) isoxazolyl,
   (R) pyrazolyl,
   (S) oxazolyl,
   (T) thiazolyl,
   (U) indolizinyl,
   (V) indazolyl,
   (W) benzothiazolyl,
   (X) benzimidazolyl,
   (Y) benzofuranyl,
   (Z) furanyl,
   (AA) thienyl,
   (BB) pyrrolyl,
   (CC) oxadiazolyl,
   (DD) thiadiazolyl,
   (FE) triazolyl,
   (FF) tetrazolyl,
   (GG) purinyl,
   (HH) 1,3-benzodioxolyl,
   (II) oxazolopyridinyl,
   (JJ) imidazopyridinyl,
   (KK) isothiazolyl,
   (LL) naphthyridinyl,
   (MM) cinnolinyl,
   (NN) carbazolyl,
   (OO) β-carbolinyl,
   (PP) isochromanyl,
   (QQ) chromanyl,
   (RR) furazanyl,
   (SS) tetrahydroisoquinoline,
   (TT) isoindolinyl,
   (UU) isobenzotetrahydrofuranyl,
   (VV) isobenzotetrahydrothienyl,
   (WW) isobenzothiophenyl,
   (XX) benzoxazolyl,
   (YY) pyridopyridinyl,
   (ZZ) benzotetrahydrofuranyl, and
   (AAA) benzotetrahydrothienyl,
wherein the R_{1-heteroaryl} group is bonded to —(CH₂)ₙ₁— by any ring atom of the parent R group substituted by hydrogen such that the new bond to the R_{1-heteroaryl} group replaces the hydrogen atom and its bond, wherein heteroaryl is optionally substituted with one thru four:
   (1) C₁-C₆ alkyl optionally substituted with one, two or three substituents selected from the group consisting of C₁-C₃ alkyl, —F, —Cl, —Br, —I, —OH, —SH, —NR₁₋ₐR_{1-b} wherein R₁₋ₐ and R_{1-b} are as defined above, —C≡N, —CF₃, C₁-C₃ alkoxy,
   (2) C₂-C₆ alkenyl with one or two double bonds,
   (3) C₂-C₆ alkynyl with one or two triple bonds,
   (4) —F, Cl, —Br and —I,
   (5) C₁-C₆ alkoxy,
   (6) —O—C₁-C₆ alkyl optionally substituted with one thru three —F,
   (7) —NR_{N-2}R_{N-3} wherein R_{N-2} and R_{N-3} are as defined above,
   (8) —OH,
   (9) —C≡N,
   (10) C₃-C₇ cycloalkyl,
   (11) —CO—(C₁-C₄ alkyl),
   (12) —SO₂—NR₁₋ₐR_{1-b} wherein R₁₋ₐ and R_{1-b} are as defined above,
   (13) —CO—NR₁₋ₐR_{1-b} wherein R₁₋ₐ and R_{1-b} are as defined above, or
   (14) —SO₂—(C₁-C₄ alkyl), with the proviso that when n₁ is zero R_{1-heteroaryl} is not bonded to the carbon chain by nitrogen;
(IV) C₂-C₆ alkenyl with one or two double bonds,
(V) C₂-C₆ alkynyl with one or two triple bonds,
(VI) —CO—NR_{N-2}R_{N-3} wherein R_{N-2} and R_{N-3} are as defined above,
(VII) —SO₂—NR_{N-2}R_{N-3} wherein R_{N-2} and R_{N-3} are as defined above,
(VIII) —CO—OH, and
(IX) —CO—O—(C₁-C₄ alkyl).

In one preferred embodiment, AA3, AA4, and AAx are natural or unnatural amino acids. The amino acids can be amino acid residues in a peptide. In another preferred embodiment, S is a solid surface such as, but not limited to, a chip, a resin, a plate, a cell surface membrane, or the like. In an alternative preferred embodiment, S is selected from the group consisting of OH, NH2, para-nitro-anilide (pNA), amido-4-methylcoumarin (AMC), and amido-4-trifluoromethylcoumarin (AFC).

In a preferred embodiment of the invention, the substrate on a solid surface S is cleaved by a solution of an enzyme thereby releasing a soluble fragment. The soluble fragment can be detected quantitatively using any analytical system or device such as, but not limited to, immunoassay, fluorometric assay, chromogenic assay, high-pressure liquid chromatography (HPLC), detection means such as colorimetry, fluorometry, radioisotope analysis, mass spectroscopy, or the like.

The invention also provides that an R-group as disclosed above may be used in any of the compounds disclosed herein that are used to identify and/or synthesize a compound having binding and/or inhibitory activity for an enzyme.

The invention contemplates compounds having particular biochemical characteristics of a peptide consensus sequence. This can be represented generally for peptide substrates of many enzymes as follows:

| | |
|---|---|
| P_{SD}-S_{SD} | Optimal substrate |
| P_{SD} alone | Poor substrate |

where S_{SD}(Secondary Specificity Determinant) is connected to the P_{SD} (Primary specificity Determinant) either in a N-terminal or C-terminal orientation by amide bonds, or may be discontinuous and exist on both sides of the P_{SD′} for example when both N-terminal and C-terminal sequences contribute to the S_{SD′. In this conception, a P}_{SD} contains at least one of the twenty naturally occurring L-amino acids, or a synthetic or unnatural amino acid (such as norleucine, ornithine, etc., for example), or a chemically modified L-amino acid (for example, F-acetyl lysine, 4-hydroxyproline, phospho-tyrosine, etc.). Catalytic conversion of a substrate with the P_{SD} intact but lacking S_{SD} may be restored (partially or completely) by a suitable small molecule organic compound that can substitute for the missing S_{SD}. This is shown schematically below, with the small molecule organic compound being represented as —R

| | |
|---|---|
| P_{SD} alone | Poor substrate |
| P_{SD} -R | Improved substrate |

with the R group being covalently attached to the P_{SD} in either a C-terminal or N-terminal orientation. Catalytic conversion would only take place if a specific R group provided the additional incremental binding affinity required to convert the necessary but insufficient binding energy provided by the P_{SD} into a catalytically sufficient binding interaction.

It follows then that if a library of chemically diverse R groups were to be covalently attached to the P_{SD} for a given enzyme, so as to generate a library of substrate-analog molecules (SAM), denoted P_{SD}—R, the SAM library could then be experimentally tested to check for improved substrate-like behavior relative to the P_{SD} alone, by mixing individual or small groups of the SAM in a suitable manner with the enzyme in question, and determining whether product formation greater than that with P_{SD} alone has taken place in any of these mixtures after incubating the mixtures for a defined period of time. The R groups present in those mixtures that have detectable product formation must therefore also be capable of binding to the enzyme effectively enough to enable catalysis.

This method therefore allows the discovery of R groups that when covalently attached to the P_{SD} can bind to the target enzyme, even though the method does not rely on having to measure a binding interaction. In fact, given the nature of product formation by enzymes (amount of product=rate of product formation×time of reaction), the procedure can be carried out at concentrations of the SAM that can be far lower than their interaction K_{d} with the enzyme target, simply by manipulating the concentrations of the target enzyme, as well as the time of incubation, while maintaining the SAM at concentrations below their limits of solubility.

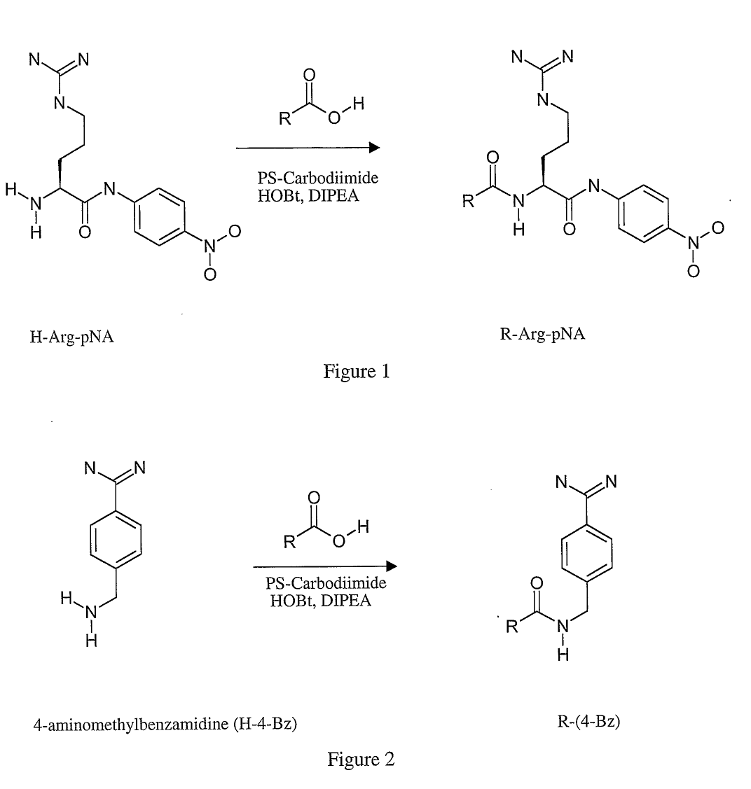

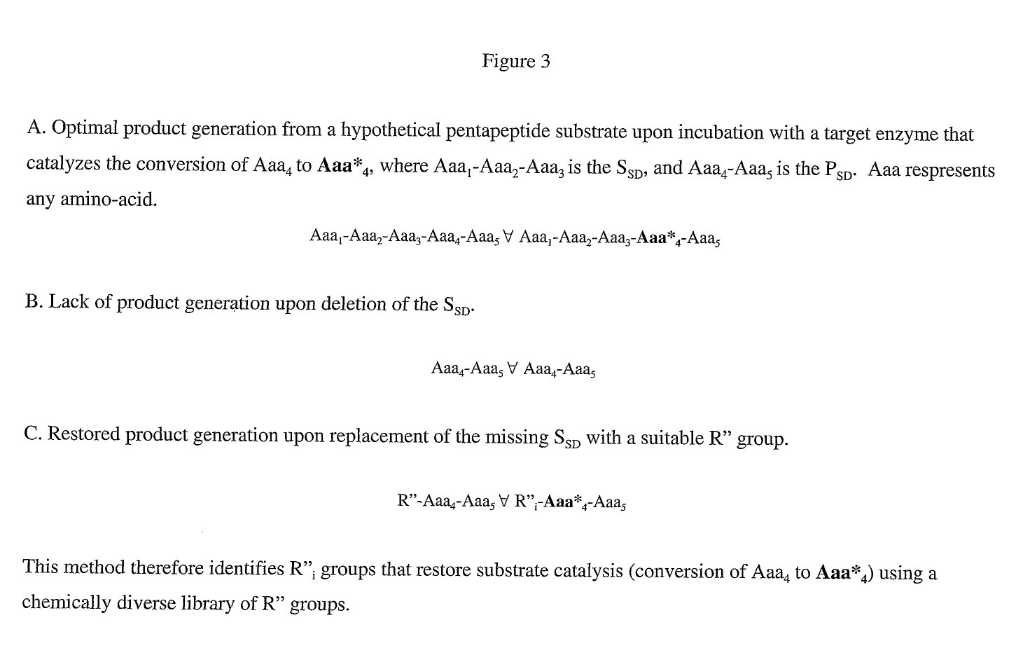

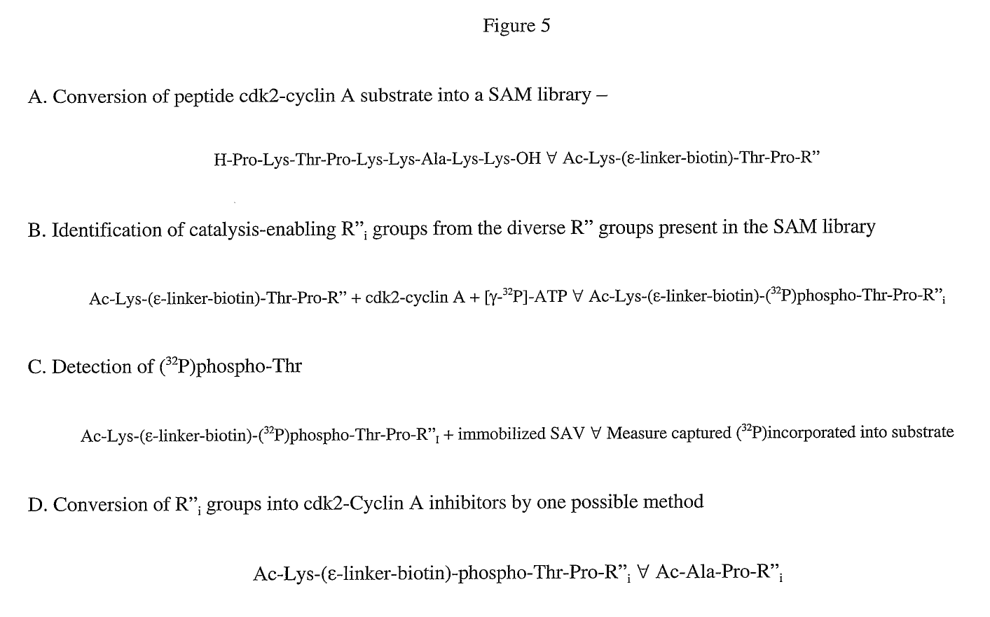

This method is broadly applicable to therapeutically important enzyme targets from many different catalytic classes, as shown in the illustrative examples in FIGS. 1 and 2. In each case, P_{SD} peptide sequences (necessary but not sufficient to enable robust catalytic conversion) are covalently attached to diverse R groups using well-known chemistry (for example, formation of amide bonds) to form chemical libraries of unique SAM, which are then tested for the ability to generate product upon incubation with the corresponding target enzyme. It is understood that elements of the substrate that must be present to detect catalytic turnover would be considered to be a part of the P_{SD}. In some cases, the P_{SD} may need to be additionally modified by attachment of non-substrate like functionalities required to facilitate the detection of product formation, as shown in the example of GSK3b and cdk2 in FIG. 5. In these examples, the chemically modified amino acid 6-(linker-biotin)-lysine is attached in an N-terminal orientation to the P_{SD} sequence (Ser/Pro), so as to enable capture with immobilized streptavidin (SAV) to facilitate detection of any incorporated radioactive phosphate during the course of a phosphorylation reaction (see Example IV, and FIG. 5).

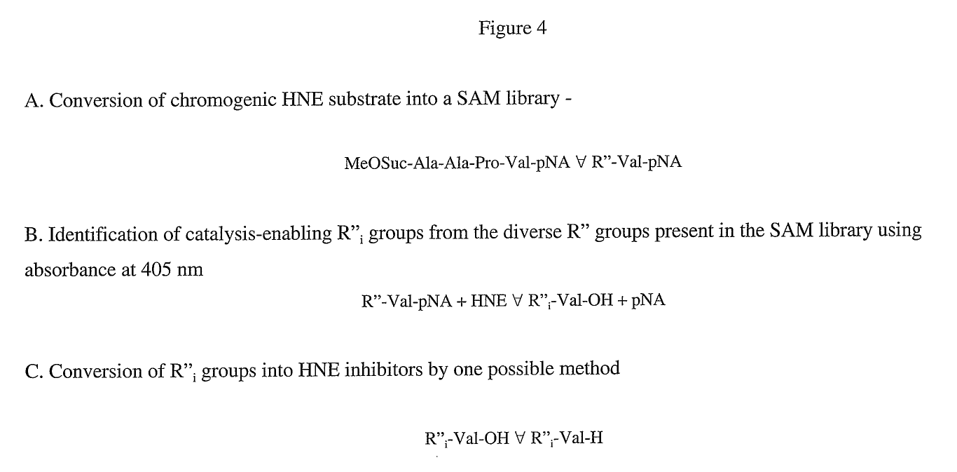

The R groups discovered by this screening method would then be considered as catalysis-enabling binders, and thus may be considered alone, or in combination with the P_{SD′} or a modified P_{SD′} to be a starting point for chemical optimization for lead discovery for the given target. New derivative SAM libraries can also be designed from the information provided by the “hits” and “non-hits” in a given ensemble of SAM, and taken through additional rounds of catalysis-enabling optimization, prior to embarking on a full-scale lead optimization campaign. Illustrative examples are provided in FIGS. 4 and 5.

The method of the invention allows one to determine if a given small molecule can bind to the active site of an enzyme, by coupling the small molecule via a suitable chemical linkage to the minimum portion of the substrate of an enzyme that is required to determine whether the chimeric molecule so constructed can be recognized as a substrate by the enzyme. Analysis of the rate of catalysis as a function of the concentration of the chimeric molecule will enable the determination of the catalytic efficiency for a given molecule, which is usually expressed as a ratio of the rate of turnover of the enzyme-substrate complex, k_{cat}, to the Michaelis constant of the formation of the enzyme-substrate complex, Kₘ. The k_{cat}/Kₘ ratio provides a quantitative measure of active site binding—with the molecules that have the most productive interactions with the active site being characterized with the highest k_{cat}/Kₘ numbers.

An example of this is provided by R—COOH groups that were used to generate chimeric substrate molecules of the form R-Arg-pNA, as shown in Table 2.

TABLE 2

| Compound | | k_{cat}/Kₘ | Relative Catalytic |
|---|---|---|---|
| Number | R—COOH | (M⁻¹s⁻¹) | Efficiency (RCE) |
| 1 | Acetic Acid | 221 | 1.0 |
| 2 | 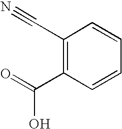 | 206 | 0.9 |
| 3 | 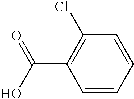 | 1,212 | 5.5 |
| 4 | 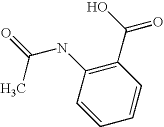 | 228 | 1.0 |
| 5 | 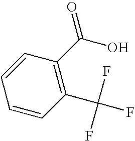 | 220 | 1.0 |
| 6 | 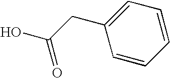 | 2,146 | 9.7 |
| 7 | 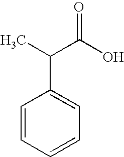 | 488 | 2.2 |
| 8 | 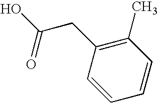 | 6,564 | 29.7 |
| 9 | 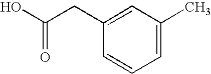 | 2,415 | 10.9 |
| 10 | 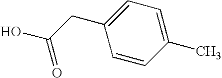 | 892 | 4.0 |

The chimeric molecules were all tested against thrombin, and their catalytic efficiencies as substrate for this enzyme determined. The k_{cat}/Kₘ values were then compared to that obtained with the reference baseline substrate Acetyl-Arg-pNA (Compound 1), which has a very low k_{cat}/Kₘ value of 221 for thrombin, under the assay conditions provided in the example. Of the four 2-substituted benzoic acids tested, Compounds 2-5, only the R group of Compound 3 (R—COOH=2-chlorobenzoic acid) enabled enhanced cleavage over baseline, and thus is selected as a compound that can productively bind to the active site of thrombin. The example is extended with the substituted phenylacetic acids utilized in Compounds 6-10, which, depending on the position of the methyl substituent, exhibit relative catalytic efficiencies varying between 2-30.

Examples of catalysis-enabling small molecules for a different enzyme, plasma kallikrein, are provided in the Table 3. Again, Compound 1, Acetyl-Arg-pNA, is used as the baseline reference point demonstrating a very low catalytic efficiency towards plasma kallikrein (k_{cat}/Kₘ=111). The R—COOH groups used for synthesizing the R-Arg-pNA in this example are based on substituted pyridine carboxylic acids (Compounds 11-15) or benzoic acids (Compounds 16-17). The selected compounds show a range of enhancement of the baseline cleavage rate, from no cleavage detected (Compound 11), to a >100-fold enhancement of the baseline cleavage rate (Compound 16).

TABLE 3

| Compound | | kcat/Km | Relative Catalytic |
|---|---|---|---|
| Number | R—COOH | (M-1s-1) | Efficiency (RCE) |
| 1 | Acetic acid | 111 | 1.0 |
| 11 | 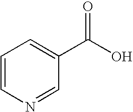 | 0 | 0.0 |
| 12 | 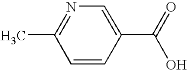 | 434 | 3.9 |
| 13 | 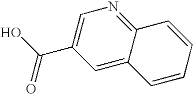 | 5,110 | 45.9 |
| 14 | 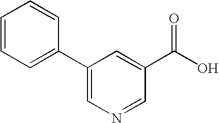 | 3,273 | 29.4 |
| 15 | 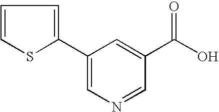 | 2,761 | 24.8 |
| 16 | 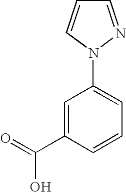 | 12,848 | 115.4 |
| 17 | 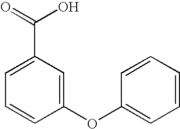 | 1,859 | 16.70 |

Thus, the method described herein provides for a very sensitive and discriminatory technique for determining the extent, if any, to which a given small molecule can make productive interactions with the active site of an enzyme. The small molecules chosen for these examples were all of the form R—COOH, to facilitate their combination with the free amino-group of H-Arg-pNA to form a stable amide linkage. However, any method that allows for the formation of a stable covalent bond between the minimal substrate portion and the small molecule can be utilized to form chimeric molecules that can then be tested. For example, molecules of the form R—CHO can be coupled to the free amino group to form chimeric alkylamine molecules of the form R—CH₂—NH-Arg-pNA, or molecules of the form R—SO₂Cl to form sulfonamides of the form R—SO₂—NH-Arg-pNA.

Although only a few examples of R—COOH have been chosen here for exemplary purposes, there is essentially no limit to the types of small molecules one could utilize to construct “libraries” of the general form R-Arg-pNA, or, for that matter, of the general form R-Aaa-pNA, where Aaa=any natural amino acid or unnatural amino acid that can serve as a primary specificity determinant for a given protease. In addition, where other enzymes or ligand-binding proteins are used for analysis, Aaa can be any natural amino acid or unnatural amino acid that can serve as a primary specificity determinant for a given enzyme or ligand-binding protein.

It is to be appreciated that different proteases have different primary specificities—the examples provided here being of serine proteases, thrombin and plasma kallikrein, that have a strong preference for the basic amino acid arginine on the amino-terminal side of the scissile bond (P1 residue) for their preferred substrates. Many other enzymes share this preference, including, but not limited to, mast cell tryptase, urokinase-type plasminogen activator (uPA), factor Xa, factor XIIa, etc, and thus libraries of the form R-Arg-pNA can be utilized to discover small molecules that can interact productively with the active sites of each of these unique enzymes. Other proteases or protease families have entirely different specificities. Thus, caspases and granzyme B share a virtually absolute requirement for aspartic acid at the P1 position of their preferred substrates, and therefore libraries of the form R-Asp-pNA can be utilized to discover small molecules that can interact productively with the active sites of such proteases. The enzyme human neutrophil elastase prefers the hydrophobic side chains of alanine, valine or leucine at the P1 position of its substrate, and thus libraries of the form R-Ala-pNA or R-Val-pNA might be utilized in such a case. In some cases, the small molecule could be attached directly to the pNA moiety, to make libraries of the form R-pNA, to discover small molecules that can be recognized by a given enzyme as an effective substitute for a known amino acid P1 residue.

Once discovered, such small molecule groups can be utilized in a variety of ways. They can be used as part of the enzyme-specific chimeric substrates, useful for detecting the presence of an enzyme in a fluid (for example, blood, plasma, urine, cerebro-spinal fluid (CSF), broncheolveolar lavage fluid, etc.) for diagnostic use. Compound 16 (Table 4) for example can be used for detecting plasma kallikrein activity (k_{cat}/Kₘ=12,848) in the presence of thrombin (k_{cat}/Kₘ=0). In contrast, Compound 8 (Table II) can be used for detecting thrombin (k_{cat}/Kₘ=6,564) in the presence of plasma kallikrein (k_{cat}/Kₘ=33). Used in combination, these two substrates can exactly determine the absolute amounts of plasma kallikrein and thrombin in a given sample where both of these enzymes may be present.

Another important use for such small molecules would be to utilize them to discover new active site-directed inhibitors of their target enzymes, to be used as possible therapeutic agents in disease conditions where the activity of the target enzyme contributes to the pathogenesis of the disease process. Thrombin, for example, is widely implicated in a variety of cardiovascular disease conditions, such as myocardial infarction, stroke, and venous thromboembolism events. Plasma kallikrein has been implicated in the pathogenesis of angioedema, and other clinically important conditions such as septic shock and inflammatory bowel disease.

One way of converting a small molecule that interacts productively at the active site of such enzymes would be combine such small molecules with ligand pharmacophores that can result in the formation of an inhibitory molecule. For example, the benzamidine moiety has been long known to those skilled in the art to be a suitable pharmacophore for binding in the pocket occupied by the P1 arginine side chain of such proteases, and can thus confer inhibitory potency when combined with other active site directed binding elements. In order to demonstrate that the small molecules that were discovered as being productive interactors with the active site could be made into inhibitors, 4-aminomethylbenzamidine (H-4-Bz) was chosen as a possible ligand pharmacophore, and molecules of the form R-(4-Bz) synthesized (FIG. 2) using the method described in Example II.

Table 4 shows examples of results obtained with R—COOH groups tested for their ability to bind to the active site of thrombin or plasma kallikrein (substrate-mode as R-Arg-pNA) and their corresponding ability to increase inhibitory potency towards thrombin or plasma kallikrein (inhibitor mode as R-(4-Bz)).

TABLE 4

| | | Plasma Kallikrein | | Thrombin | |
|---|---|---|---|---|---|
| Compound | | R-Arg-pNA | R-(4-Bz) | R-Arg-pNA | R-(4-Bz) |
| Number | R—COOH | k_{cat}/Kₘ (M⁻¹s⁻¹) | Kᵢ (μM) | k_{cat}/Kₘ (M⁻¹s⁻¹) | Kᵢ (μM) |
| 1 | Acetic acid | 111 | 85.4 | 221 | 114.7 |
| 18 | 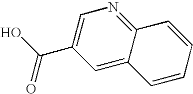 | 5,110 | 1.1 | −8 | 116.1 |
| 19 | 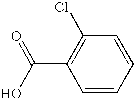 | 356 | 17.0 | 1,212 | 22.2 |
| 20 | 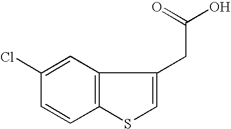 | 1,113 | 3.4 | 1,802 | 6.6 |
| 21 | 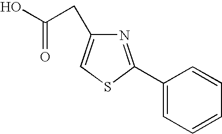 | 323 | 15.3 | 16,472 | 7.6 |

Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N,N-dimethylformamide. The peptide is cleaved between the peptide carboxy terminus and the linker group to yield a peptide acid or amide. (Novabiochem 1997/98 Catalog and Peptide Synthesis Handbook, San Diego Calif. pp. S1-S20). Automated synthesis may also be carried out on machines such as the ABI 431A peptide synthesizer (PE Biosystems). A protein or portion thereof may be substantially purified by preparative high performance liquid chromatography and its composition confirmed by amino acid analysis or by sequencing (Creighton (1984) Proteins, Structures and Molecular Properties, W H Freeman, New York N.Y.).In another embodiment, a pharmaceutical composition comprising a compound having binding activity to an enzyme in conjunction with a pharmaceutical carrier may be administered to a subject to treat or prevent a condition associated with altered expression or activity of the enzyme including, but not limited to, those provided above. Preferably the compound has inhibitory activity to the enzyme. The pharmaceutical composition may be employed in ingested, parenteral, implanted, injected, transdermal, transmucosal, device coating and other drug delivery applications.

### Pharmacology

Pharmaceutical compositions are those substances wherein the active ingredients are contained in an effective amount to achieve a desired and intended purpose. The determination of an effective dose is well within the capability of those skilled in the art. For any compound, the therapeutically effective dose may be estimated initially either in cell culture assays or in animal models. The animal model is also used to achieve a desirable concentration range and route of administration. Such information may then be used to determine useful doses and routes for administration in humans. Pharmaceutically acceptable refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability.

A therapeutically effective dose refers to that amount of protein or inhibitor that ameliorates the symptoms or condition. Therapeutic efficacy and toxicity of such agents may be determined by standard pharmaceutical procedures in cell cultures or experimental animals, for example, ED₅₀ (the dose therapeutically effective in 50% of the population) and LD₅₀ (the dose lethal to 50% of the population). The dose ratio between toxic and therapeutic effects is the therapeutic index, and it may be expressed as the ratio, LD₅₀/ED₅₀. Pharmaceutical compositions that exhibit large therapeutic indexes are preferred. The data obtained from cell culture assays and animal studies are used in formulating a range of dosage for human use.

### Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

### Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess potential consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism. Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted: (1) an initial dose-range-finding experiment, (2) an experiment to narrow the range of effective doses, and (3) A Final Experiment for Establishing the Dose-Response Curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, having a duration of a year or more, are used to demonstrate either the absence of toxicity or the carcinogenic potential of an agent. When studies are conducted on rats, a minimum of three test groups plus one control group are used, and animals are examined and monitored at the outset and at intervals throughout the experiment.

### EXAMPLES

The invention will be more readily understood by reference to the following examples, which are included merely for purposes of illustration of certain aspects and embodiments of the present invention and not as limitations.

### Example I

### Synthesis of R-Arg-pNA Compounds

The following protocol was utilized for the synthesis of all compounds of the general form R-Arg-paranitroanilide (R-Arg-pNA), starting from R—COOH and H-Arg-pNA, utilizing resin-bound carbodiimide reagent (PS-Carbodiimide) and hydroxybenzotriazole (HOBt) (See FIG. 1). R—COOH were obtained from commercial suppliers (ASDI, Maybridge, U.K. or ASDI Biosciences, Newark Del.). PS-Carbodiimide was obtained from Biotage (Charlottesville Va.). H-Arg-pNA was obtained from either Bachem Bio Science Inc (King of Prussia Pa.) or Chem-Impex International Inc. (Wood Dale Ill.). HOBt was obtained from Anaspec (San Jose Calif.). Methylene chloride (DCM) was obtained from JT Baker (Phillipsburg N.J.). Dimethylsulfoxide (DMSO) was obtained from Pierce Chemical (Rockford Ill.). Diisopropylethylamine (DIPEA) was obtained from Aldrich (St Louis Mo.).

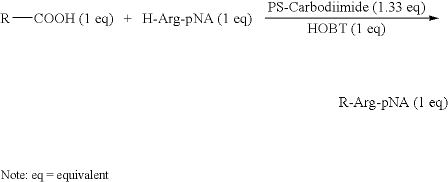

Aliquots of PS-Carbodiimide resin (100 μmols) were dispensed into inlet-closed, fritted, polypropylene cartridges, and mixed with 1 ml of DCM each. Each cartridge then received 75 μmols of an individual R—COOH (2.5 ml of 30 mM R—COOH solution in 25% DMSO/75% DCM, followed by 1 ml of 75 mM H-Arg-pNA.2HCl solution in DMSO, 75 μl of a 1M solution of HOBt in DMSO, and 13 μl of DIPEA. The cartridges were stoppered firmly, placed on an Adams Nutator, and mixed at room temperature for 72 hours. The inlets were then opened, and individual reaction mixtures drained into separate 20 ml glass vials. The remaining resins were washed with 2.5 ml of 25% DMSO/75% DCM, and the washings combined with the reaction mixtures in the glass vials. DCM and DIPEA were removed by evaporation, with the R-Arg-pNA product obtained as a DMSO solution.

The amount of R-Arg-pNA present in solution was determined by (a) measuring the amount of free H-Arg-pNA left in the DMSO solutions, and subtracting this amount from (b) the total amount of pNA present in the DMSO solution.

The amount of free H-Arg-pNA left in the DMSO solution was determined as follows: 5 μl aliquots of the DMSO solutions obtained after evaporation were mixed with 45 μl of DMSO in individual wells of a 96-well plate, followed by the addition of 50 μl of a 1:1 solution of 3% ninhydrin in DMSO: 4 M sodium acetate, pH 5.0. The plate was incubated at 37° C. for 1 hour, and the A₄₅₀ in individual wells recorded in a Molecular Devices Vmax Plate Reader (Molecular Devices, Sunnyvale, Calif.). The A₄₅₀ was determined to be proportional to the amount of H-Arg-pNA present in solution, established using wells containing known concentrations of H-Arg-pNA which produced a relationship of the form

[H-Arg-pNA]=B[A₄₅₀]+C,
where B and C are constants that were empirically determined for each assay from a standard curve.

This allowed the concentration of H-Arg-pNA left in individual reaction mixtures ([H-Arg-pNA]_{R}) to be calculated by measuring the A₄₅₀ in individual wells and then using the equation above to solve for [H-Arg-pNA]_{R}. The calculations were carried out using the SOFTMAX PRO data acquisition and analysis program provided by Molecular Devices (Sunnyvale Calif.).

The concentration of total pNA-containing product in solution for a given R-Arg-pNA ([pNA]_{R}) was determined as follows: 2 μl aliquots of the individual DMSO solutions of R-Arg-pNA were mixed with 198 μl of an aqueous solution of 1 M NaOH, in individual wells of a 96-well plate. The hydrolysis of the Arg-pNA amide bond at the elevated pH led to the release of free pNA, with an accompanying increase in A₄₀₅. The reaction was complete within 3 h at 37° C., and the A₄₀₅ in individual wells recorded in a Molecular Devices Vₘₐₓ Plate Reader. The concentration of pNA in each sample was calculated by comparing the A₄₀₅ of the corresponding well to a standard curve generated by using known concentrations of H-Arg-pNA (0, 5, 10, 20, 40 mM).

The concentration of the desired product in each sample is then obtained as follows—

[R-Arg-pNA]=[pNA]_{R}−[H-Arg-pNA]_{R}

### Example II

### Synthesis of R-(4-Bz) Compounds

The following protocol was utilized for the synthesis of all compounds of the general form R-(4-Bz), starting from R—COOH and 4-aminomethylbenzamidine (H-4-Bz), utilizing resin-bound carbodiimide reagent (PS-Carbodiimide) and hydroxybenzotriazole (HOBt) (See FIG. 2). H-4-Bz was obtained from Astatech (Bristol Pa.).

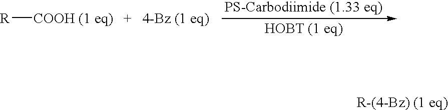

Aliquots of PS-Carbodiimide (Biotage) resin (100 μmols) were dispensed into inlet-closed, fritted, polypropylene cartridges, and mixed with 0.75 ml of DCM each. Each cartridge then received 75 μmols of an individual R—COOH (2.5 ml of 30 mM R—COOH solution in 25% DMSO/75% DCM) followed by 1 ml of 75 mM H-4-Bz solution in DMSO, 75 μl of a 1M solution of HOBt in DMSO, and 13 μl of DIPEA. The cartridges were stoppered firmly, placed on an Adams Nutator, and mixed at room temperature for 72 hours. The inlets were then opened, and individual reaction mixtures drained into separate 20 ml glass vials. The remaining resins were washed with 2.5 ml of 25% DMSO/75% DCM, and the washings combined with the reaction mixtures in the glass vials. DCM and DIPEA were removed by evaporation, with the end products in the reaction mixture obtained as a DMSO solution.

The concentration of R-4-Bz obtained was determined by (a) measuring the amount of free H-4-Bz left in the reaction mixture, and subtracting this amount from (b) the total amount of H-4-Bz recovered in a control reaction mixture to which no R—COOH group was added. The concentration of free H-4-Bz left in the reaction mixture was determined as follows: 5 μl aliquots of the DMSO solutions obtained after evaporation were mixed with 45 μl of DMSO in individual wells of a 96-well plate, followed by the addition of 50 μl of a 1:1 solution of 3% ninhydrin in DMSO:4 M sodium acetate, pH 5.0. The plate was incubated at 37° C. for 15 minutes, and the A₄₅₀ in individual wells recorded in a Molecular Devices Vₘₐₓ Plate Reader. The A₄₅₀ was determined to be proportional to the amount of H-4-Bz present in solution, established using wells containing known concentrations of H-4-Bz, which produced an equation of the form

[H-4-Bz]=B[A₄₅₀]+C,
where B and C represent constants that were empirically determined for each assay.

This allowed the concentration of H-4-Bz left in individual reaction mixtures ([H-4-BZ]_{R}) to be calculated by measuring the A₄₅₀ in individual wells and then using the equation above to solve for [H-4-BZ]_{R}. The calculations were carried out using the SOFTMAX PRO data acquisition and analysis program provided by Molecular Devices.

[R-4-Bz]=[H-4-BZ]_{control}−[H-4-BZ]_{R},
where [H-4-BZ]_{R} represents the concentration of free H-4-Bz left in a given reaction mixture.

### Example III

### Determination of Cleavage of R-Arg-pNA

Proteases can perform the following reaction:

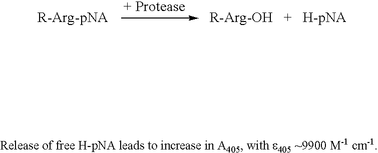

R-Arg-pNA stock solutions (10-30 mM) were adjusted to 2 mM by dilution with DMSO. 10 μl aliquots of each were then mixed with 90 μl of a buffered solution of 50 mM HEPES, pH 8, containing 5% DMSO and 0.01% Triton X-100, in individual wells of a 96-well plate. The solutions were mixed by gentle agitation and then were set aside for 60 minutes at room temperature. A solution of the protease (human plasma kallikrein (Enzyme Research Laboratories, Swansea, U.K.) or human thrombin (Haemtech, Dural, NSW, Australia)) was made up in the same buffer at 2× the final concentration to be attained in the assay (5 nM for plasma kallikrein, 40 nM for thrombin), and 100 μl of the enzyme solution was added to each of the R-Arg-pNA-containing wells. The plate was then read in a kinetic mode in the Molecular Devices Vₘₐₓ plate reader at 405 nm, at 12-15 seconds intervals for up to 30 minutes. The rate of increase in A₄₀₅ in any given well was converted into rate of substrate cleavage.

### Example IV

### Determination of Enzyme Inhibition by R-(4Bz)

R-(4-Bz) stock solutions (10-30 mM) were adjusted to 0.4 mM by dilution with DMSO. 5 μl aliquots of each were then mixed with 95 μl of 50 mM HEPES, pH 8, containing 5% DMSO, and 0.1 mM of a suitable chromogenic substrate, in individual wells of a 96-well plate. For plasma kallikrein, the chromogenic substrate was Z-Phe-Arg-pNA, and for thrombin it was Z-Pro-Arg-pNA. The solutions were mixed by gentle agitation and then set aside for 60 min at room temperature. A solution of the enzyme (plasma kallikrein or thrombin) was made up in the same buffer at 2× the final concentration to be attained in the assay (1 nM plasma kallikrein, 10 nM thrombin), and 100 μl of the enzyme solution was added to each of the wells, so as to attain final concentrations of 0.01 mM R-(4-Bz) and 0.05 mM of the chromogenic substrate. The plate was then read in a kinetic mode in the Vₘₐₓ plate reader at 405 nm, at 15 second intervals for up to 15 minutes. The rate of substrate cleavage in wells containing R-(4Bz) were compared to control wells (5 μl DMSO), and any decrease was converted into “% inhibition”, according to the following formula—

% inhibition=1−Rate_{R-(4Bz)}/Rate_{DMSO}

Kᵢ values for inhibition were estimated by the following relationship

Kᵢ=[R-(4-Bz)]/((Rate_{DMSO}/Rate_{R-(4-Bz)})−1)

### Example V

### SAM Library for the Serine Protease Human Neutrophil Elastase

Human neutrophil elastase (HNE) is a serine protease that preferentially cleaves peptide bonds C-terminal to the small aliphatic amino acids Ala or Val (Bieth et al. Handbook of Proteolytic Enzymes, 2^{nd} Ed., pp 1517-1523, Elsevier (2004)). Thus, it will cleave the synthetic tetrapeptide chromogenic substrate MeOSuc-Ala-Ala-Pro-Val-pNA efficiently, cleavage being accompanied by the release of free pNA, with an increase in its characteristic absorbance at 405 nm (A₄₀₅). Replacement of valine (Val) with other amino acid residues, such as arginine (Arg), leads to a dramatic loss of substrate turnover, so, for this substrate, Val-pNA can be designated as a P_{SD} for BNE, and MeOSuc-Ala-Ala-Pro as the S_{SD}. To generate a SAM library for BNE, R groups of the general formula R-COOH can be covalently attached to H-Val-pNA by using PS-Carbodiimide/HOBt as described in Example I, leading to the formation of compounds of the form R-Val-pNA. The R—COOH groups can be selected using specific criteria from commercially available or synthetically feasible organic molecules. For example, the R groups could be selected using the following general search criteria from the MDL Available Chemicals Database (MDL-ACD, Elsevier): MW≦300, # of proton acceptors ≦5, # of protein donors ≦2, etc. It is understood that other general or specific selection criteria could also be utilized to determine the composition of the R groups, and thus the exact composition of the SAM, and it is not the intention of this example to limit the chemical nature of the R group. Individual compounds from the SAM library of R-Val-pNA thus obtained would then be incubated with BNE at a suitable concentration in a buffer such as 50 mM HEPES, pH 7.5, 0.1% Triton X-100, containing 5% DMSO. Positive (MeoSuc-Ala-Ala-Pro-Val-pNA) and negative (Ac-Val-pNA) controls for HNE activity would also be separately assayed to determine the dynamic range of the assay method. An increase in A₄₀₅ over time in a given incubation mixture, after correcting for any background cleavage of the negative control, would then be taken as a measure of catalysis-enabling binding of the particular R in that specific SAM, and therefore be classified as a positive “hit”. Such groups could then be utilized to design an inhibitor of FNE, of the form R-Val-H, for example (FIG. 4).

### Example VI

### SAM Library for the Serine/Threonine Kinase Cdk2-Cyclin A

The heterodimeric complex cdk2-cyclin A, formed between the cyclin-dependent kinase cdk2 and cyclin A, functions as a catalytically competent Ser/Thr kinase during the progression of the cell cycle. Cdk2-cyclin A has an absolute specificity for proline at the P₊₁ residue, thus can be considered to have Ser/Thr-Pro as its P_{SD}. A well-characterized peptide substrate for cdk2-cyclin A is H-Pro-Lys-Thr-Pro-Lys-Lys-Ala-Lys-Lys-Leu-OH (Stevenson-Lindert et al, (2003) J. Biol. Chem., 278: 50956), with the P_{SD} sequence shown in bold. In this example, the S_{SD} information resides mostly on the flanking C-terminal side of the P_{SD}. In this case, a SAM library can be designed of the following general form, Ac-Lys(ε-linker-biotin)-Thr-Pro-R, where R groups of the general form NH₂—R are attached covalently to the C-terminal end of the Ac-Lys(ε-linker-biotin)-Thr-Pro-OH, using PS-Carbodiimide/HOBt as in Example I. Individual compounds from this unique SAM library are then incubated with catalytically active cdk2-cyclin A, in the presence of [γ³²P]-ATP. After a suitable period of time, Streptavidin-Agarose is added to the samples, leading to the capture of the biotinylated SAM molecules. The samples are filtered and washed with a suitable buffer to remove all free [γ³²P]-ATP, then the radioactivity immobilized on the Streptavidin-Agarose measured using a suitable detection device. The radioactivity immobilized in any given sample is directly proportional to the extent of phosphorylation of the unique compound present in that sample. Positive (Ac-Lys-(ε-linker-biotin)-Thr-Pro-Lys-Lys-Ala-Lys-Lys-Leu) and baseline (Ac-Lys(ε-linker-biotin)-Thr-Pro-NH₂) controls for cdk2-cyclin A activity are separately assayed as well to determine the dynamic range of the assay method. An increase in the incorporation of [ε³²P] into an individual SAM over that in the baseline control is then taken as a measure of the catalysis-enabling binding of the particular R group present in that SAM. Such groups could then be utilized to design an inhibitor of cdk2-cyclin A, of the form Ac-Ala-Pro-R, for example (FIG. 5).

### Example VII

### SAM Library for the Protein Tyrosine Phosphatase PTP-1b

The human phosphotyrosine protein phosphatase PTP-1b has been implicated in the etiology of diabetes and Alzheimer's disease. A SAM library for PTP-1b is constructed by coupling R—COOH to H-(p)Tyr-NH₂, using PS-Carbodiimide/HOBt as in Example I, so as to obtain compounds of the form R-(p)Tyr-NH₂. Incubation of a suitable amount of PTP-1b with such compounds in a suitable buffer, such as 50 mM HEPES, pH 7.5, is carried out to determine whether an enhanced rate of dephosphorylation is obtained with a given R-group compared to the baseline rate of Ac-(p)Tyr-NH₂ dephosphorylation. The extent of dephosphorylation with time is determined by monitoring the absorbance at 282 nm using a kinetic plate-reader, since dephosphorylation of phosphotyrosine to tyrosine results in an increase in A282 (Zhang et al. (1993) Anal. Biochem. 211: 7-15). R-groups that provide a RCE>2 are coupled to, for example, H-difluorophosphonate-tyrosine-NH₂, a nonhydrolyzable phosphotyrosine analog, to obtain inhibitors of PTP-1b.

Although the various exemplary embodiments of the present invention are directed to uses as disclosed herein, the present invention is not limited to such uses, and the method described may be used for any application in which it is important to synthesize a compound that interacts with another compound.

Those skilled in the art will appreciate that various adaptations and modifications of the just-described embodiments can be configured without departing from the scope and spirit of the invention and the above description is intended to be illustrative, and not restrictive, and it is understood that the applicant claims the full scope of any claims and all equivalents.

1. A method for identifying a compound having binding activity for an enzyme, the method comprising the steps of:
(i) providing an enzyme;
(ii) incubating a first sample of the enzyme with a first substrate thereby creating a first incubate, the first substrate comprising at least one R-group and wherein the enzyme catalyses conversion of the first substrate into a first product;
(iii) measuring the increase in product formation,
(iv) converting the rate of increase of product formation into a rate of first substrate catalysis by the enzyme (Rate_{first});
(v) incubating a second sample of the enzyme with a second substrate thereby creating a second incubate, the second substrate comprising at least one P_{SD} but not an R-group and wherein the enzyme catalyses conversion of the second substrate into a second product;
(vi) measuring the change in second product formation,
(vii) converting the change of product formation into a rate of second substrate catalysis by the enzyme (Rate_{second});
(viii) determining the k_{cat}/Kₘ ratio of the first substrate (k_{cat}/Kₘ first);
(viii) determining the k_{cat}/Kₘ ratio of the second substrate (k_{cat}/Kₘ second); and
(ix) determining the relative catalytic efficiency (RCE) of the R group; wherein a first substrate with a RCE>2 is identified as a compound having binding activity for the enzyme.

2. The method of claim 1 wherein the enzyme is selected from the group consisting of proteases, kinases, phosphatases, hydrolases, oxidoreductases, isomerases, transferases, methylases, acetylases, ligases, and lyases.

3. The method of claim 1, wherein the P_{SD} comprises a moiety selected from the group consisting of an amino acid residue, an oligopeptide, a saccharide, a polysaccharide, a lipid, a phospholipid, a fatty acid, a glycoproterin, a proteoglycan, an aminoglycan, an alcohol; amine, a nucleoside, a nucleotide, an oligonucleotide, a glycosyl phosphatidyl inositol, and a steroid.

4. The method of claim 1, wherein the second substrate further comprises a moiety selected from the group consisting of an amino acid residue, an oligopeptide, a saccharide, a polysaccharide, a lipid, a phospholipid, a fatty acid, a glycoproterin, a proteoglycan, an aminoglycan, an alcohol amine, a nucleoside, a nucleotide, an oligonucleotide, a glycosyl phosphatidyl inositol, and a steroid.

5. The method of claim 1 wherein measuring the rate of increase of product formation is selected from the group consisting of using analytical means to measure change in optical density of the incubate, colorimetry, fluorimetry, mass-spectroscopy, radioisotope analysis, pH analysis, phase partition of product, and electrochemical analysis of product.

6. The method of claim 1 wherein the first substrate further comprises a bond selected from the group consisting of an amide bond, a peptide bond, a covalent bond, a double bond, a triple bond, a keto bond, an oxo bond, a disulfide bond, and a phosphate bond.

7. The method of claim 1 wherein the incubate is incubated at a temperature from about 4° C. to about 75° C.

8. (canceled)

9. (canceled)

10. The method of claim 7 wherein the temperature is from about 16° C. to about 25° C.

11. The method of claim 1 wherein the RCE is determined using the formula RCE=(k_{cat}/Kₘ first)/(k_{cat}/Kₘ second).

12. The method of claim 1 wherein the R group of the compound is less than 5000 daltons (Da) in size, has logP<15.0, has hydrogen bond donors <15, and has hydrogen bond acceptors <30.

13. (canceled)

14. The method of claim 12 wherein the R group of the compound is less than 500 Da in size, has logP<5.0, has hydrogen bond donors <5, and has hydrogen bond acceptors <10.

15. A method for identifying a compound having inhibitory activity for an enzyme, the method comprising the steps of:
(i) providing an enzyme;
(ii) incubating a first sample of the enzyme with a first substrate thereby creating a first incubate, the first substrate comprising at least one R-group and wherein the enzyme catalyses conversion of the first substrate into a first product;
(iii) measuring the increase in first product formation,
(iv) converting the rate of increase of product formation into a rate of first substrate catalysis by the enzyme (Rate_{first});
(v) providing a second substrate, the second substrate comprising at least one P_{SD} but not an R-group;
(vi) incubating a second sample of the enzyme with the second substrate thereby creating a second incubate, wherein the enzyme catalyses conversion of the second substrate into a second product;
(vii) measuring the increase in second product formation in the second incubate; X
(viii) converting the rate of first product formation into a rate of first substrate catalysis by the enzyme in the presence of second substrate (Rate_{second});
(ix) incubating a compound and another sample of the second substrate thereby creating a third incubate, the compound comprising at least one R-group;
(x) adding a third sample of the enzyme to the third incubate;
(xi) incubating the third incubate;
(xii) measuring the increase in second product formation in the third incubate;
(xiii) converting the rate of second product formation into a rate of second substrate 1 catalysis by the enzyme in the presence of the compound (Rate_{compound});
(xiv) determining the k_{cat}/Kₘ ratio of the first substrate (k_{cat}/Kₘ first);
(xv) determining the k_{cat}/Kₘ ratio of the second substrate (k_{cat}/Kₘ second); and
(xvi) determining the relative catalytic efficiency (RCE) of the R-group; wherein the first substrate and the compound have at least one R-group in common, and wherein an R-group having a RCE>2 is identified as a compound having inhibitory activity for the enzyme.

16. The method of claim 15 wherein the enzyme is selected from the group consisting of proteases, kinases, phosphatases, hydrolases, oxidoreductases, isomerases, transferases, methylases, acetylases, ligases, and lyases.

17. The method of claim 15 further comprising the step of determining the inhibition constant (Kᵢ) of the compound using the formula Kᵢ=[compound]/((Rate_{second}/Rate_{compound})−1).

18. The method of claim 17 wherein the K; is between about 0.1 μM and 200 μM.

19. (canceled)

20. (canceled)

21. (canceled)

22. The method of claim 17 wherein the K; is less than 85 μM.

23. The method of claim 15 wherein the R group of the compound is less than 5000 daltons (Da) in size, has logP<15.0, has hydrogen bond donors <15, and has hydrogen bond acceptors <30.

24. (canceled)

25. The method of claim 24 wherein the R group of the compound is less than 500 Da in size, has logP<5.0, has hydrogen bond donors <5, and has hydrogen bond acceptors <10.

26. The method of claim 15 wherein the inhibitory activity of the compound is selected from the group consisting of reversible inhibition and irreversible inhibition.

27. A method for identifying a small organic molecule (R group) having binding activity to a target enzyme, the method comprising the steps of:
(i) synthesizing a compound comprising an R group and a synthetic peptide (SP) having the general formula R—SP or SP—R, the R group and the synthetic peptide linked using a covalent bond, wherein the synthetic peptide portion of the resulting molecule comprises a P_{SD} for the target enzyme, the synthetic peptide comprising at least one amino acid residue (Aaa) and one peptide bond;
(ii) mixing the synthetic compound with the target enzyme under conditions that allow the target enzyme to have sufficient catalytic activity upon the synthetic compound;
(iii) measuring the amount of product generated;
(iv) determining the rate of product formation (Rate_{synthetic compound});
(v) comparing the rate of product formation with a rate of product formation generated from a reaction comprising the target enzyme and another substrate (Rate_{other substrate}), the other substrate selected from the group consisting of a natural substrate, a chromogenic substrate, a fluorogenic substrate, and a modified substrate; and
(vi) determining the RCE of the synthetic compound by RCE=(Rate_{synthetic compound}/Rate_{other substrate}), wherein if the RCE is >2, the small organic molecule R group is identified as an active-site binder of the target enzyme.

28. The method of claim 27, wherein the R group is selected from an organic molecule having at least one carboxylic acid group.

29. The method of claim 27, wherein the R group is selected from an organic molecule having at least one primary or secondary amino group.

30. The method of claim 27, wherein the SP is of the form selected from the group consisting of H-Aaa-para-nitroanilide (H-Aaa-pNA), H-Aaa-7-amido-4-methylcoumarin (H-Aaa-AMC), H-Aaa-7-amido-4-trifluoromethylcoumarin (H-Aaa-AFC), H-Aaa₁-Aaa₂-X, H-Aaa₁-Aaa₂-Aaa₃-X, H-Aaa₁-Aaa₂-Aaa₃-Aaa₄-X, H-Aaa₁-Aaa₂-Aaa₃-Aaa₄-Aaa₅-X, Aaa₁-Aaa₂-Aaa₃-Aaa₄-Aaa₅-X, Y-Aaa₁-Aaa₂-OH, Y-Aaa₁-Aaa₂-Aaa₃-OH, Y-Aaa₁-Aaa₂-Aaa₃-Aaa₄-OH, and Y-Aaa₁-Aaa₂-Aaa₃-Aaa₄-Aaa₅-OH, where Aaa, Aaa₁, Aaa₂, Aaa₃, Aaa₄ and Aaa₅ are selected from the group consisting of any one of the twenty naturally occurring L-amino acids, a synthetic amino acid, an unnatural amino acid, and a chemically modified L-amino acid, where X is selected from the group consisting of OH, NH₂, pNA, AMC, and AFC, and Y is selected from the group consisting of H and CH₃C(═O).

31. (canceled)

32. (canceled)

33. (canceled)

34. (canceled)

35. (canceled)

36. (canceled)

37. (canceled)

38. (canceled)

39. (canceled)

40. (canceled)

41. The method of claim 27, wherein the target enzyme is a protease.

42. The method of claim 27, wherein the target enzyme is a protein kinase.

43. The method of claim 27, wherein the target enzyme is a protein phosphatase.

44. The method of claim 27, wherein the target enzyme is a proline hydroxylase.

45. The method of claim 27, wherein the target enzyme is a histone deacetylase.

46. The method of claim 30, wherein the Aaa is selected from the group consisting of: Arg, Lys, Pro, Asp, Val, Cys, and Tyr.

47. (canceled)

48. (canceled)

49. The method of claim 30, wherein the synthetic peptide is of the form selected from the group consisting of H-Ser-Pro-Lys-X, H-Thr-Pro-Lys-X, Y-Lys-Ser-Pro-OH, and Y-Lys-Thr-Pro-OH, where X is OH or NH₂, and Y is H or CH₃C(═O).

50. (canceled)

51. (canceled)

52. (canceled)
